(19) United States
(12) Patent Application Publication
(10) Pub. No.: US 2026/0196293 A1
(43) Pub. Date: Jul. 9, 2026

(54) DIFFUSION MODEL FOR GENERATIVE PROTEIN DESIGN

(71) Applicant: Generate Biomedicines, Inc., Somerville, MA (US)

(72) Inventor: John Ingraham, Cambridge, MA (US)

(73) Assignee: Generate Biomedicines, Inc., Somerville, MA (US)

(21) Appl. No.: 19/128,154

(22) PCT Filed: Nov. 8, 2023
(86) PCT No.: PCT/US2023/037034

§ 371 Date: May 7, 2025

Related U.S. Application Data
(60) Provisional application No. 63/423,775, filed on Nov. 8, 2022.
(60) Provisional application No. 63/383,074, filed on Nov. 9, 2022.
(60) Provisional application No. 63/424,044, filed on Nov. 9, 2022.
(60) Provisional application No. 63/383,242, filed on Nov. 10, 2022.
(60) Provisional application No. 63/384,076, filed on Nov. 16, 2022.
(60) Provisional application No. 63/385,020, filed on Nov. 26, 2022.
(60) Provisional application No. 63/385,619, filed on Nov. 30, 2022.
(60) Provisional application No. 63/499,963, filed on May 3, 2023.
(60) Provisional application No. 63/469,822, filed on May 30, 2023.
(60) Provisional application No. 63/470,672, filed on Jun. 2, 2023.
(60) Provisional application No. 63/522,538, filed on Jun. 22, 2023.
(60) Provisional application No. 63/578,763, filed on Aug. 25, 2023.

(51) Int. Cl. G16B 15/20; G06F 40/205; G16B 15/30; G16B 40/20

(52) U.S. Cl. CPC G16B 15/20; G16B 15/30; G16B 40/20; G06F 40/205

(57) ABSTRACT

A system is disclosed for de novo protein generation. The system receives a set of design condition(s) that specify target characteristics of a synthetic protein. The system defines a modular energy function as a composition of a diffusion energy component and one or more conditioner energy components. The system applies a diffusion model to determine a denoised protein backbone. In applying the diffusion model, in each sampling step: the system transforms one prior sampled state of the synthetic protein from unconstrained space into constrained space based on the one or more design conditions, denoises the prior sampled state in the constrained space, and samples a subsequent sampled stated by applying a gradient of the modular energy function to the denoised prior sampled state in the constrained space. The final sampled state is a denoised protein backbone for the synthetic protein that satisfies the set of design condition(s).

### CROSS-REFERENCE TO RELATED APPLICATIONS

The present application claims the benefit of and priority to U.S. Provisional Application No. 63/423,775 filed on Nov. 8, 2022, U.S. Provisional Application No. 63/424,044 filed on Nov. 9, 2022, U.S. Provisional Application No. 63/383,074 filed on Nov. 9, 2022, U.S. Provisional Application No. 63/383,242 filed on Nov. 10, 2022, U.S. Provisional Application No. 63/384,076 filed on Nov. 16, 2022, U.S. Provisional Application No. 63/385,020 filed on Nov. 26, 2022, U.S. Provisional Application No. 63/385,619 filed on Nov. 30, 2022, U.S. Provisional Application No. 63/499,963 filed on May 3, 2023, U.S. Provisional Application No. 63/469,822 filed on May 30, 2022, U.S. Provisional Application No. 63/470,672 filed on Jun. 2, 2023, U.S. Provisional Application No. 63/522,538 filed on Jun. 22, 2023, and U.S. Provisional Application No. 63/578,763 filed on Aug. 25, 2023, all of which are incorporated by reference in their entirety.

### BACKGROUND

Challenges arise when trying to design protein as protein space is vast. Because of this vast space, modeling the relationship between amino acid sequences, protein structure, and function is extremely difficult. Some computational techniques to iteratively sample and explore the protein space have been implemented, but such techniques are ill-equipped with traversing the vast protein space as computations remain combinatorially large. Moreover, attempting to discover de novo protein sequences that satisfy particular design conditions often lead to models pigeonholing into a small subset of the protein space, typically around prior known protein sequences.

### SUMMARY

An analytics system implements a diffusion model for generative protein design. The analytics system receives a set of one or more design conditions for generating a de novo protein. The diffusion model is guided by the set of design conditions to conditionally sample the protein space in generating the de novo protein. In one or more embodiments, the analytics system generates a modular energy function that drives the conditional sampling of the protein space. The modular energy function thereby constrains the sampling process to satisfy the design conditions. Design conditions are effectively target characteristics of the desired protein to be designed. During deployment, the diffusion model is configured to denoise from an initial random state in the protein space to determine the de novo protein. In some embodiments, the analytics system may also determine the protein residue sequence, the protein folding structure, or some combination thereof.

To train the diffusion model, the analytics system leverages known proteins from a protein database. The analytics system injects Gaussian noise to the proteins to generate noised states of the proteins. The analytics system applies the diffusion model to the noised states to predict the denoised states of the proteins. The analytics system then trains the diffusion model to minimize a loss determined by comparing the predicted denoised states to the initial states of the proteins. When training, the analytics system may directly predict the denoised state of the training sample, thereby scaling subquadratically.

In some embodiments, the analytics system implements low-temperature sampling to target high likelihood regions in the multidimensional protein space. The low-temperature sampling algorithm implements low temperature rescaling and hybrid Langevin dynamics to better guide the diffusion process towards the high-likelihood distributions, i.e., optima in the protein space. The low-temperature rescaling aims to exploit high likelihood states, whereas the equilibration rate of the Langevin dynamics operates as a counterbalance to promote exploration of the protein space.

With the novel synthetic protein design, the analytics system may provide the design to a protein manufacturing system to manufacture the synthetic protein. In general, the protein manufacturing system uses synthesized DNA molecules coded for the expression of the amino acid sequence of the synthetic protein. The manufacturing system transfects a cell line with the synthetically generated DNA molecules. Example cell lines include bacteria, yeast, and mammalian cells. The transfected cell lines are cultivated to express the protein through the cell's natural functions. Following protein expression, the manufacturing system may perform protein extraction and purification to yield a high-quality and functional protein product. The end result is the extracted and purified synthetic protein. Thus, the disclosure includes a synthetic protein that is generated by a process that includes the steps presented below. However, in some embodiments, the system provides or designs a data representation of a synthetic protein. Thus, the disclosure includes a synthetic protein representation or a synthetic protein design that is generated by or designed by a process includes the steps presented below. The synthetic protein can be a novel or de novo protein that does currently exist or has not previously existed in nature or that is not currently known to exist in nature, or that has not previously been discovered or not known to have been discovered in nature.

Clause 1. A computer-implemented method comprising: receiving a set of one or more design conditions that specify target characteristics of a synthetic protein; defining a modular energy function as a composition of a diffusion energy component and one or more conditioner energy components, wherein the diffusion energy component determines an energy value based on a sampled state of the synthetic protein and a time step of the sampled state and each conditioner energy component determines an energy value based on the sample state of the synthetic protein and the target characteristic of each design condition; and applying a diffusion model to determine a denoised protein backbone, wherein applying the diffusion model comprises, in each sampling step of a plurality of sampling steps: transforming one prior sampled state of the synthetic protein from unconstrained space into constrained space based on the one or more design conditions, denoising the prior sampled state in the constrained space, and sampling a subsequent sampled state in the unconstrained space by applying a gradient of the modular energy function to the denoised prior sampled state in the constrained space; wherein the final sampled state is a denoised protein backbone for the synthetic protein that satisfies the set of one or more design conditions.

Clause 2. The computer-implemented method of clause 1, wherein each design condition is either: a restraint that reweights the modular energy function to bias for a target characteristic of the synthetic protein; or a constraint that limits multidimensional protein space that defines possible states of the synthetic protein.

Clause 3. The computer-implemented method of any of clauses 1-2, wherein the set of one or more design conditions one or more of: a symmetry constraint that requires symmetry in the denoised protein backbone; a substructure infilling restraint that biases towards particular substructures; a shape constraint that requires a particular shape of the denoised protein backbone; a distance constraint that requires a particular distance between at least two residues; a substructure root mean squared deviation (RMSD) constraint that requires a structural motif to have a low RMSD; a text caption restraint derived from a text input including one or more design conditions; a sequence constraint that requires the denoised protein backbone to include a particular amino acid sequence; a domain classifier constraint that inputs a target structure and outputs a functional characteristic required of the denoised protein backbone; and a secondary structure constraint that requires a particular secondary structure to be present in the denoised protein backbone.

Clause 4. The computer-implemented method of any of clauses 1-3, further comprising: applying a sequence generation model to the denoised protein backbone to determine an amino acid sequence that folds into the denoised protein backbone.

Clause 5. The computer-implemented method of any of clauses 1-4, wherein the diffusion model is further configured to output an amino acid sequence that is configured to structurally create the denoised protein backbone.

Clause 6. The computer-implemented method of any of clauses 1-5, wherein an initial state is a base protein backbone to be modified by the diffusion model and is input with the set of one or more design conditions.

Clause 7. The computer-implemented method of any of clauses 1-5, wherein an initial state is randomly sampled in multidimensional protein space.

Clause 8. The computer-implemented method of any of clauses 1-7, wherein the plurality of sampling steps are discretized timesteps.

Clause 9. The computer-implemented method of clause 8, wherein the plurality of sampling steps includes 100 or more sampling steps.

Clause 10. The computer-implemented method of any of clauses 1-9, wherein the set of one or more design conditions are derived from applying a natural language processing model to an input text query.

Clause 11. The computer-implemented method of any of clauses 1-10, wherein, in each sampling step, sampling another sampled state comprises rescaling the modular energy function based on a time-dependent temperature.

Clause 12. The computer-implemented method of clause 11, wherein, in each sampling step, sampling another sampled state comprises applying a time-dependent Langevin dynamics equilibration rate.

Clause 13. The computer-implemented method of any of clauses 1-12, further comprising: initializing a first seed state and a second seed state that is different than the first seed state; wherein applying the diffusion model comprises applying the diffusion model to the first seed state to determine a first denoised protein backbone and applying the diffusion model to the second seed state to determine a second denoised protein backbone.

Clause 14. The computer-implemented method of any of clauses 1-13, further comprising: receiving a second set of one or more design conditions that specify one or more modifications to the denoised protein backbone of the synthetic protein; modifying the modular energy function to further comprise one or more conditioner energy components based on the second set of one or more design conditions; and applying the diffusion model to modify the denoised protein backbone to satisfy the one or more modifications to the denoised protein backbone.

Clause 15. A non-transitory computer-readable storage medium storing instructions that, when executed by a computer processor, cause the computer processor to perform the computer-implemented method of any of clauses 1-14.

Clause 16. A system comprising: a computer processor; and the non-transitory computer-readable storage medium of clause 15.

Clause 17. A non-transitory computer-readable storage medium storing a synthetic protein design that is generated by the computer-implemented method of any of clauses 1-14.

Clause 18. A synthetic protein that is generated by a process comprising steps of: determining a synthetic protein design that is generated by the computer-implemented method of any of clauses 1-14; and manufacturing the synthetic protein via cell expression.

Clause 19. A computer-implemented method for training a diffusion model, comprising: accessing from a protein database a set of protein backbones; generating a noised state for each protein backbone by transforming an initial state of the protein backbone with noise; applying the diffusion model to the noised state for each protein backbone to predict a denoised state of the protein backbone; determining a loss for each protein backbone as a difference between the denoised state and the initial state of the protein backbone; and training the diffusion model as a neural network by adjusting one or more parameters of the diffusion model based on the losses.

Clause 20. The computer-implemented method of clause 19, wherein a protein backbone comprises three-dimensional coordinates for each heavy atom of amino acid residues in the protein backbone.

Clause 21. The computer-implemented method of any of clauses 19-20, wherein generating the noised state for each protein backbone comprises, for each protein backbone: selecting a random time step on a time continuum, wherein the initial state is at time step zero; and adding an amount of Gaussian noise based on the random time step to the initial state of the protein backbone to generate the noised state.

Clause 22. The computer-implemented method of clause 21, wherein applying the diffusion model to the noised state for each protein backbone comprises: predicting the amount of Gaussian noise added to generate the noised state based on the initial state and the random time step; and removing the predicted amount of Gaussian noise from the noised state to generate the denoised state.

Clause 23. The computer-implemented method of any of clauses 21-22, further comprising: generating a second noised state for each protein backbone by: selecting a second random time step on the time continuum, and adding an amount of Gaussian noise based on the second random time step to the initial state of the protein backbone to generate the second noised state; and applying the diffusion model to the second noised state for each protein backbone to predict a second denoised state of the protein backbone; determining a second loss for each protein backbone as a difference between the second denoised state and the initial state of the protein backbone; and wherein training the diffusion model is further based on the second losses.

Clause 24. The computer-implemented method of any of clauses 19-23, wherein the loss for each protein backbone is based on a difference between coordinates of each heavy atom of amino acid residues in the denoised state and coordinates of each heavy atom of amino acid residues in the initial state.

Clause 25. The computer-implemented method of any of clauses 19-24, further comprising: filtering the protein database to deduplicate similar protein backbones.

Clause 26. The computer-implemented method of clause 25, wherein filtering the protein database to deduplicate similar protein backbones comprises: determining a similarity score between a first protein backbone and a second protein backbone as a distance between coordinates of the first protein backbone and coordinates of the second protein backbone; and removing the second protein backbone based on the similarity score being below a threshold.

Clause 27. The computer-implemented method of any of clauses 19-26, further comprising: filtering the protein database to obtain a high percentage of protein backbones of one type of protein.

Clause 28. A computer program product comprising: a non-transitory computer-readable storage medium storing a diffusion model generated by the computer-implemented method of any of clauses 19-27.

Clause 29. A non-transitory computer-readable storage medium storing a diffusion model generated by the computer-implemented method of any of clauses 19-27.

Clause 30. A system comprising: a computer processor; and the non-transitory computer-readable storage medium of clause 29.

Clause 31. A computer-implemented method comprising: receiving an input with an inverse temperature and an equilibration rate; generating an energy function comprising a diffusion energy component which determines an energy value based on a state of a synthetic protein; modifying a reverse-time dynamics function with a first scaling factor based on the inverse temperature, wherein the reverse-time dynamics function comprises a gradient of the energy function; modifying a Langevin dynamics function with a second scaling factor based on the equilibration rate, wherein the Langevin dynamics function comprises the gradient of the energy function; generating an aggregate dynamics function by combining the modified reverse-time dynamics function and the modified Langevin dynamics function; initializing an initial state of a protein backbone comprising coordinates of heavy atoms of amino acids of a synthetic protein; applying a diffusion model to the initial state to determine a denoised protein backbone, wherein applying the diffusion model comprises, in each sampling step of a plurality of sampling steps: denoising one prior sampled state, and sampling a subsequent sampled stated by applying the aggregate dynamics function to the denoised prior sampled state; wherein the final sampled state is the denoised protein backbone for the synthetic protein.

Clause 32. The computer-implemented method of clause 31, wherein the inverse temperature is configured to drive the sampling towards high-likelihood regions of multidimensional protein space.

Clause 33. The computer-implemented method of any of clauses 31-32, wherein the equilibration rate is a ratio of Langevin dynamics to conventional dynamics.

Clause 34. The computer-implemented method of any of clauses 31-33, wherein the initial state is randomly sampled in multidimensional protein space.

Clause 35. The computer-implemented method of any of clauses 31-34, wherein the plurality of sampling steps are discretized timesteps of a time continuum.

Clause 36. The computer-implemented method of clause 35, wherein the plurality of sampling steps includes 100 or more sampling steps.

Clause 37. The computer-implemented method of any of clauses 31-36, further comprising: applying a sequence generation model to the denoised protein backbone to determine an amino acid sequence that folds into the denoised protein backbone.

Clause 38. The computer-implemented method of any of clauses 31-37, wherein the diffusion model is further configured to output an amino acid sequence that is configured to structurally create the denoised protein backbone.

Clause 39. The computer-implemented method of any of clauses 31-38, wherein an initial sampled state is a base protein backbone to be modified by the diffusion model and is input with the inverse temperature and the equilibration rate.

Clause 40. The computer-implemented method of any of clauses 31-38, wherein an initial sampled state is randomly sampled in multidimensional protein space.

Clause 41. A non-transitory computer-readable storage medium storing instructions that, when executed by a computer processor, cause the computer processor to perform the computer-implemented method of any of clauses 31-40.

Clause 42. A system comprising: a computer processor; and the non-transitory computer-readable storage medium of clause 41.

Clause 43. A non-transitory computer-readable storage medium storing a synthetic protein design that is generated by the computer-implemented method of any of clauses 31-40.

Clause 44. A synthetic protein that is generated by a process comprising steps of: determining a synthetic protein design that is generated by the computer-implemented method of any of clauses 31-40; and manufacturing the synthetic protein via cell expression.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a system environment of an analytics system implementing a diffusion model for generative protein design, according to one or more embodiments.

FIG. 2 is a block diagram of the analytics system implementing the diffusion model, according to one or more embodiments.

FIG. 3 illustrates a training process of the diffusion model, according to one or more embodiments.

FIG. 4 illustrates deployment of the diffusion model to generate a protein backbone based on a set of one or more design conditions, according to one or more embodiments.

FIG. 5 is a block diagram exampling the architecture of a diffusion model, according to one or more embodiments.

FIG. 6 is a block diagram exampling the architecture of a backbone graph neural network, according to one or more embodiments.

FIG. 7 is a block diagram exampling the architecture of a sequence generation model, according to one or more embodiments.

FIG. 8 illustrates a flowchart describing training of a diffusion model for protein design, according to one or more embodiments.

FIG. 9 illustrates a flowchart describing de novo protein generation through deployment of a diffusion model, according to one or more embodiments.

FIGS. 10A-10C illustrate Hybrid Langevin SDE to sample from temperature-perturbed distributions, according to one or more example implementations.

FIG. 11 illustrates representative samples identified using this modified SDE for low-temperature sampling, according to one or more example implementations.

FIG. 12 illustrates various structural characteristics of synthetic protein designs generated with the diffusion model, according to one or more example implementations.

FIG. 13 illustrates synthetic protein designs generated with the diffusion model, according to one or more example implementations.

FIG. 14A illustrates conditioning on arbitrary symmetry groups is possible by symmetrizing gradient, noise, and initialization through the sampling process, according to one or more example implementations.

FIG. 14B illustrates conditioning on partial substructure (monochrome) enables protein “infilling” or “outfilling,” according to one or more example implementations.

FIG. 14C illustrates conditioning on arbitrary volumetric shapes by using gradients derived from Optimal Transport, according to one or more example implementations.

FIG. 14D illustrates further conditioning based on other various design conditions, according to one or more example implementations.

### DETAILED DESCRIPTION

### Overview

One of the cornerstone technical challenges in novel protein design is exploring the vast multidimensional protein space. Past approaches have been limited in their success in exploring the protein space. Reasons for this include 1) modeling the relationship between sequence, structure, and function is difficult, and 2) most computational design methods rely on iterative search and sampling processes which must navigate a rugged fitness landscape incrementally. Due to the vastness of the protein space, these iterative search and sampling processes may be limited to exploring already known designs. Determining how to efficiently explore the space of designable protein structures remains an open challenge.

Here, an analytics system implements a diffusion model that accelerates the exploration of the protein space through a learned reverse diffusion process. The learned diffusion process is trained efficiently by learning the instantaneous reverse-time diffusion process with training samples. The training thereby provides an improvement to the technical field of computation protein design. During deployment, the diffusion model may be conditioned through one or more design conditions to generate novel protein designs that satisfy target characteristics specified by the design conditions. Such provides flexibility in the protein design process to utilize the same learned diffusion process to generate distinct, diverse, yet novel protein designs. A user may further modify protein designs based on the conditioner framework, allowing for tailored designs. Further, in some embodiments, the system implements a low-temperature sampling algorithm that modifies the dynamics to guide the diffusion process towards high-likelihood distributions, also amounting to a technical improvement. All the above amount to technical improvements and practical applications in the field.

With the novel synthetic protein design, the analytics system may provide the design to a protein manufacturing system to manufacture the synthetic protein. In general, the protein manufacturing system uses synthesized DNA molecules coded for the expression of the amino acid sequence of the synthetic protein. The manufacturing system transfects a cell line with the synthetically generated DNA molecules. Example cell lines include bacteria, yeast, and mammalian cells. The transfected cell lines are cultivated to express the protein through the cell's natural functions. Following protein expression, the manufacturing system may perform protein extraction and purification to yield a high-quality and functional protein product. The end result is the extracted and purified synthetic protein. Accordingly, the diffusion model may be applied to create real-world physical synthetic proteins, e.g., not previously found in nature. Such is a practical application.

The training of the machine-learned models described herein (such as the diffusion models, neural networks, and other models referenced herein) include the performance of one or more non-mathematical operations or implementation of non-mathematical functions at least in part by a machine or computing system, examples of which include but are not limited to data loading operations, data storage operations, data toggling or modification operations, non-transitory computer-readable storage medium modification operations, metadata removal or data cleansing operations, data compression operations, protein structure modification operations, image modification operations, noise application operations, noise removal operations, and the like. Accordingly, the training of the machine-learned models described herein may be based on or may involve mathematical concepts, but is not simply limited to the performance of a mathematical calculation, a mathematical operation, or an act of calculating a variable or number using mathematical methods.

Likewise, it should be noted that the training of the models describes herein cannot be practically performed in the human mind alone. The models are innately complex including vast amounts of weights and parameters associated through one or more complex functions. Training and/or deployment of such models involve so great a number of operations that it is not feasibly performable by the human mind alone, nor with the assistance of pen and paper. In such embodiments, the operations may number in the hundreds, thousands, tens of thousands, hundreds of thousands, millions, billions, or trillions. Moreover, the training data may include hundreds, thousands, tens of thousands, hundreds of thousands, millions, or billions of protein backbones (or derivatives thereof), each protein backbone may further include hundreds, thousands, tens of thousands, hundreds of thousands, or millions of three-dimensional coordinates of heavy atoms in the peptide sequence. Accordingly, such models are necessarily rooted in computer-technology for their implementation and use.

### System Environment

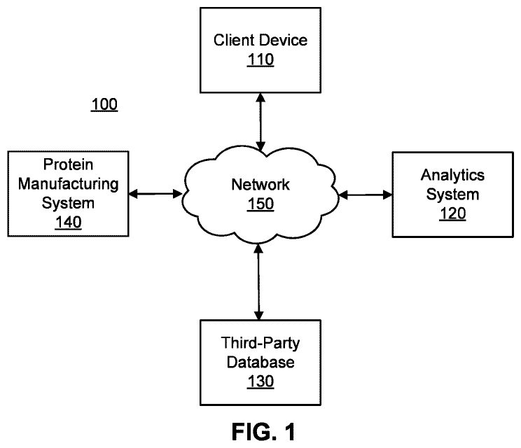

FIG. 1 illustrates an example system environment for an analytics system 130, in accordance with one or more embodiments. The system environment illustrated in FIG. 1A includes a client device 110, an analytics system 120, a third-party database 130, a protein manufacturing system 140, and a network 150. Alternative embodiments may include more, fewer, or different components from those illustrated in FIG. 1, and the functionality of each component may be divided between the components differently from the description below. Additionally, each component may perform their respective functionalities in response to a request from a human, or automatically without human intervention.

A client device 110 may be operated by a user in designing proteins. The client device 110 is configured to receive inputs and to display results of analyses by the analytics system, including synthetic protein designs. Accordingly, the client device 110 is a computing device that interacts with other components in the system environment 100 via the network 140. In one or more embodiments, a user may provide to the client device 110 an input including a set of one or more design conditions, optionally with any other instructions, for generating one or more synthetic proteins. The client device 110 may relay the input to the analytics system 120 via the network 140 to generate the one or more synthetic proteins. After generation of the synthetic proteins, the analytics system 120 may relay the generated synthetic proteins to the client device 110 for display to the user. The user may provide additional inputs to the client device 110 to modify the generated protein designs or to regenerate protein designs.

In one or more embodiments, the client device 110 may present a design interface for protein design. The design interface may accept input in various forms. For example, the design interface may include a set of togglable menus. Each togglable menu may include target characteristics for the target protein. In one example, a first togglable menu may include all types of symmetry. Another togglable menu may include different structural motifs to include. A third togglable menu may include various protein types (e.g., antibodies, contractile proteins, enzymes, hormonal proteins, structural proteins, storage proteins, and transport proteins). Other types of input options in the design interface may include range inputs, text input, sequence input, picture input, etc. For example, the design interface may include a single text input for inputting a text string.

The analytics system 120 performs one or more computational analyses. The analytics system 120 is configured to receive inputs from the client device 110 to guide protein design. The analytics system 120 generally applies a diffusion model in conjunction with a sampling algorithm to generate a de novo protein design. The de novo protein design may be provided to a manufacturing system for manufacturing of the protein. The analytics system 120 may also provide the de novo protein design to the client device 110, e.g., for display in the design interface. The client device 110 may provide further inputs for modification of the protein design.

In one or more embodiments, the analytics system 120 generates protein design with a set of one or more design conditions that constrain the protein design. The inputs from the client device 110 may include the set of one or more design conditions including target characteristics of a protein to be generated. The analytics system 120 utilizes the design conditions to constrain application of the diffusion model. The analytics system defines a modular energy function based on the one or more design conditions. The analytics system also transforms coordinates of the sampled state from unconstrained space into constrained space based on the one or more design conditions. The analytics system traverses the protein space from an initial sampled state. At each sampling step, the analytics system utilizes the diffusion model to determine a subsequent sampled state based on the modular energy function and the one or more design conditions. The final sampled state is a protein backbone, e.g., defined by three-dimensional coordinates of residue heavy atoms in the protein chain.

In one or more embodiments, the analytics system 120 may generate a full amino acid sequence configured to structurally create the protein backbone. In one or more embodiments, the diffusion model may be trained to further output the full amino acid sequence. In other embodiments, the analytics system 120 deploys a sequence generation model to determine the full amino acid sequence. The sequence generation model inputs the protein backbone and outputs the full amino acid sequence.

Prior to deployment of the diffusion model, the analytics system 120 may train the diffusion model. The analytics system 120 retrieves protein backbones for use as training samples, e.g., from a database. With each protein backbone, the analytics system transforms an initial state of the protein backbone into a noised state by injecting noise. The amount of noise injected is based on random sampling of a time step on a time continuum. At training time, the analytics system 120 applies the diffusion model to the noised states of the training samples to predict a denoised state from the noised state. The denoised state is predicted based on a gradient of an energy function as applied to the noised state. The analytics system 120 may determine a loss for each training sample based on the initial state and the denoised state. The analytics system 120 trains the diffusion model, e.g., by adjusting parameters (also referred to as weights) of the diffusion model, to minimize the losses.

In one or more embodiments, the analytics system 120 leverages low-temperature sampling during deployment of the diffusion model to drive the sampling towards high-likelihood and confident regions. The low-temperature sampling algorithm implements low temperature rescaling and hybrid Langevin dynamics to better guide the diffusion process towards high-likelihood distributions, i.e., optima in the protein space. The low temperature rescaling may include a combination of a temperature-adjusted reverse time stochastic differential equation (SDE) and a temperature-adjusted probability flow ordinary differential equation (ODE). The hybrid Langevin dynamics may include a combination of an annealed Langevin dynamics SDE and a Langevin reverse-time SDE.

The third-party database 130 is an online database that stores data, e.g., that may be retrieved and used by the analytics system 120. In one or more embodiments, the third-party database 130 stores data on past protein designs. Each protein design may be described by a nucleic acid sequence coded for expression of the protein, an amino acid sequence of the protein (and variants thereof), information on protein folding structure, protein function, chemical properties, physical properties, thermodynamic properties, etc.

The protein manufacturing system 140 is a platform for manufacturing protein. In some embodiments, the protein manufacturing system 140 may be a human-operated laboratory environment. In other embodiments, the protein manufacturing system 140 may be an automated platform with one or more devices for manufacturing protein. For example, the protein manufacturing system 140 may include a DNA synthesis device for manufacturing DNA molecules for coding a target protein. The DNA synthesis device may implement chemical synthesis to create the DNA molecules. Chemical synthesis is a solid-phase phosphoramidite chemical process. In chemical synthesis, the desired DNA sequence is built step-by-step by adding one nucleotide at a time. The process occurs on a solid support, usually a controlled pore glass bead, where the first nucleotide is attached. The synthesis proceeds using a series of reactions to add each subsequent nucleotide successively. This method can produce DNA molecules, e.g., up to 200 base pairs long. These synthesized DNA molecules can be assembled into larger constructs. The protein manufacturing system 140 may also include another protein synthesis device for protein expression with the synthetically generated DNA molecules coded for expression of the target protein. The protein synthesis device may be configured to transfect a cell line with the synthetically generated DNA molecules. Example cell lines include bacteria, yeast, and mammalian cells. The choice of host cell system depends on factors such as scalability, cost, and compatibility with the protein's structure and function. The transfected cell lines are maintained to produce the protein through the cell's natural functions. Following protein expression, the protein manufacturing system 140 may perform protein extraction and purification to yield a high-quality and functional protein product. Common purification methods include affinity chromatography, ion exchange chromatography, size exclusion chromatography, and precipitation. The end result is the extracted and purified target protein.

In some embodiments, the protein manufacturing system 140 may also perform one or more wet lab analyses on the protein manufactured. Wet lab analyses aim to characterize or to validate the manufactured protein. For example, the protein manufacturing system 140 may sequence the manufactured protein to determine whether the manufactured protein matches to the intended target protein. In other examples, the protein manufacturing system 140 may characterize the structure of the manufactured protein, e.g., through x-ray crystallography. The protein manufacturing system 140 may further run experiments with the manufactured protein while measuring characteristics, e.g., denaturing the manufacture protein to determine refolding structure, etc.

The client device 110, the analytics system 120, the third-party database 130, and the protein manufacturing system 140 can communicate with each other via the network 150. The network 150 is a collection of computing devices that communicate via wired or wireless connections. The network 150 may include one or more local area networks (LANs) or one or more wide area networks (WANs). The network 150, as referred to herein, is an inclusive term that may refer to any or all of standard layers used to describe a physical or virtual network, such as the physical layer, the data link layer, the network layer, the transport layer, the session layer, the presentation layer, and the application layer. The network 150 may include physical media for communicating data from one computing device to another computing device, such as multiprotocol label switching (MPLS) lines, fiber optic cables, cellular connections (e.g., 3G, 4G, or 5G spectra), or satellites. The network 150 also may use networking protocols, such as TCP/IP, HTTP, SSH, SMS, or FTP, to transmit data between computing devices. In some embodiments, the network 150 may include Bluetooth or near-field communication (NFC) technologies or protocols for local communications between computing devices. The network 150 may transmit encrypted or unencrypted data.

### Analytics System

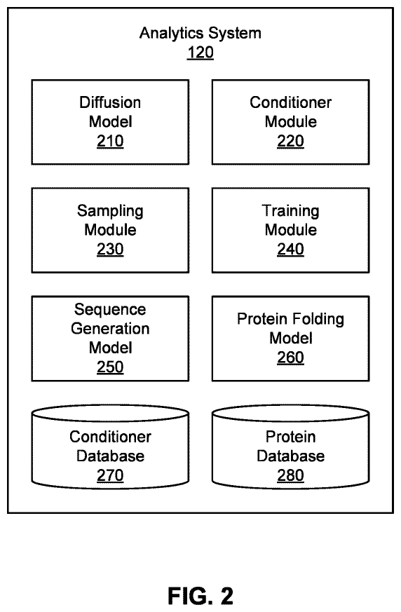

FIG. 2 is a block diagram of the analytics system 120 implementing a diffusion model for de novo protein generation, according to one or more embodiments. The analytics system 120 includes the diffusion model 210, a conditioner module 220, a training module 230, and a sampling module 240, a sequence generation model 250, a protein folding model 260, a conditioner database 270, and a protein database 280. In other embodiments, the analytics system 120 may have additional, fewer, or different components than those listed in FIG. 2.

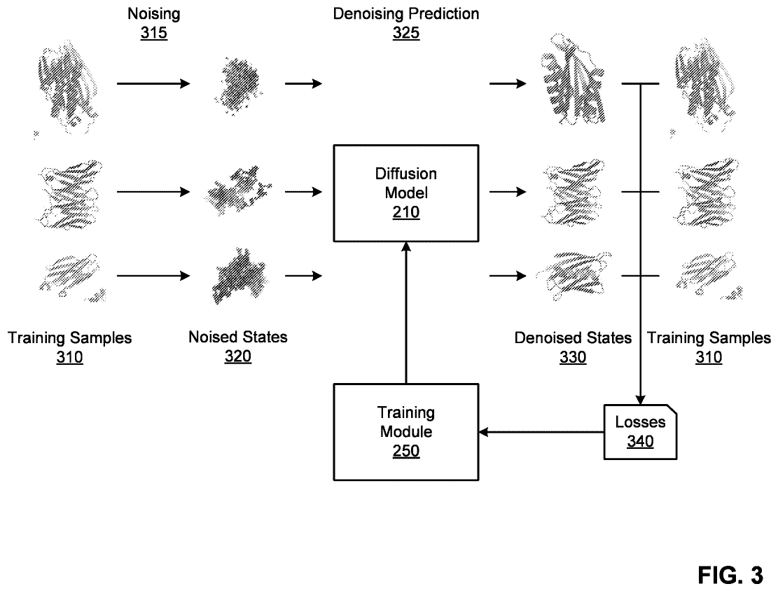

The diffusion model 210 is configured to transform one state of a protein backbone into another state of the protein backbone through removal of noise. The diffusion model 210 is a computation, machine-learning generative model. The diffusion model 210 simulates the forward and reverse diffusion of a protein backbone on a time continuum, i.e., where t∈[0, 1]. The denoised state is at time step t=0, whereas time step t=1 represents complete diffusion and thereby loss of any signal. When training the diffusion model 210, the diffusion model 210 learns to predict the reverse flow of time, from a noised state (at some time step in the time continuum) to the denoised state at time step t=0. At run-time, the diffusion model 210 is configured to predict small steps of reverse diffusion that is guided by a sampling algorithm employed by the sampling module 230. The diffusion model 210 inputs one state and outputs another state based on the input state and an energy function. In one or more embodiments, the energy function is a modular energy function defined by a diffusion energy component and one or more conditioner energy components. The diffusion energy component is a baseline energy component, whereas the conditioner energy components further modify the energy function to satisfy the one or more design conditions. Further details relating to the diffusion model 210 are described below in conjunction with FIGS. 3-6.

The conditioner module 220 conditions deployment of the diffusion model 210 based on a set of one or more design conditions. Design conditions are target characteristics of a target protein. The design conditions may include one or more restraints and one or more constraints. The restraints are soft conditions that bias the diffusion model to achieve a target characteristic. The constraints are hard conditions that limit the multidimensional protein space to ensure target protein wholly satisfies the constraints. Example design conditions include, but are not limited to, a symmetry constraint, a substructure infilling restraint, a shape constraint, a distance constraint, a substructure root mean squared deviation (RMSD) constraint, a text caption restraint, a sequence constraint, a domain classifier constraint, a secondary structure constraint, etc. The symmetry constraint specifies a certain symmetry of the target protein. The substructure infilling restraint biases towards particular substructures. The shape constraint specifies a particular shape of the target protein. The distance constraint specifies a particular distance between at least two residues. The substructure RMSD constraint specifies a structural motif to have a low RMSD. The text caption restraint biases towards a text input including one or more design conditions. The sequence constraint specifies the target protein to include a particular amino acid sequence. The secondary structure constraint specifies a particular secondary structure to be present in the target protein. In one or more embodiments, the conditioner module 220 applies a natural language processing (NLP) model to parse a text query into the one or more design conditions. The NLP model may be machine-learning model.

In one or more embodiments, the conditioner module 220 generates the modular energy function based on the set of one or more design conditions. The conditioner module 220 may generate the modular energy function by selecting a baseline conditioner energy component for each design condition. The conditioner module 220 may further modify the conditioner energy component based on the design condition. For example, the conditioner energy component for the distance constraint may include a variable for the distance value specified. Accordingly, the conditioner module 220 fills in the variable of the baseline conditioner energy component with the specified distance value. In other examples, the symmetry constraint design condition may include different conditioner energy components for each type of possible symmetry. Further details relating to different design conditions are described below in the subsection entitled “Conditioner Examples.”

The sampling module 230 implements a sampling algorithm to traverse the multidimensional protein space with the diffusion model 210 to generate a de novo protein. Initially, the sampling module 230 may determine the initial sampled state. The initial sampled state may be a random state in the multidimensional protein space. The sampling module 230 iteratively inputs one sampled state into the diffusion model 210 to generate another sampled state based on the modular energy function as applied to the first sampled stated. The sampling module 230 iterates over a plurality of sampling steps. In one or more embodiments, the number of sampling steps is based on a time increment. For example, if the sampling module initializes a random state at t=0.850 on the time continuum that ranges t∈[0, 1], with to =0 as the time step of the initial state, then the number of sampling steps is 850 with a time increment Δt=0.001. The final sampled state where t=0 is the de novo protein backbone.

In some embodiments, the sampling module 230 may leverage a constrained space defined by the set of one or more design conditions. The sampling module 230 transforms an input sampled state in unconstrained space into constrained space based on the one or more design conditions. The sampling module 230 may denoise the input state in the constrained space. The sampling module 230 may transform the denoised input state from the constrained space back into the unconstrained space. Then the sampling module 230 may apply the gradient of the diffusion model's modular energy function to the input sampled state (e.g., after denoising in the constrained space, and transformed back into the unconstrained space) to determine an output sampled state.

In some embodiments, the sampling module 230 performs low-temperature sampling. The low-temperature sampling algorithm implements low temperature rescaling and hybrid Langevin dynamics to better guide the diffusion process towards high-likelihood distributions, i.e., optima in the protein space. The low temperature rescaling may include a combination of a temperature-adjusted reverse time stochastic differential equation (SDE) and a temperature-adjusted probability flow ordinary differential equation (ODE). The hybrid Langevin dynamics may include a combination of an annealed Langevin dynamics SDE and a Langevin reverse-time SDE. Further details relating to different design conditions are described below in the subsection entitled “Low-Temperature Sampling.”

The training module 240 trains the diffusion model 210. The training module 240 may train the diffusion model to predict a reverse-time diffusion process of protein backbones. The training module 240 may obtain a set of known protein backbones to use as training samples for training the diffusion model 210. The known protein backbones may be accessed from the third-party database 130. For each known protein backbone's initial state, the training module 240 may inject an amount of noise based on a randomly sampled time step on the time continuum. For example, the time continuum ranges t∈[0, 1], with to =0 as the time step of the initial state and tₙ is the randomly selected time step on the time continuum. At t=1, all signal is lost, and the protein backbone is completely noised. The greater the time step, the more noise is injected into the training sample. The training module 240 trains the diffusion model 210 to predict the initial state from the noised state. To accomplish the training, the training module 240 applies the diffusion model 210 to the noised state to predict the denoised state. The training module 240 calculates, for each training sample, a loss between the initial state and the predicted denoised state. The training module 240 tunes the diffusion model 210, i.e., by adjusting parameters of the diffusion model 210, to minimize the losses of the training samples. As the training module 240 trains the diffusion model 210 by directly predicting the initial state, the training scales [symbol] (N). Further details related to training of the diffusion model 210 are described in conjunction with FIG. 3.

The sequence generation model 250 generates a full amino acid sequence for a de novo protein backbone. The sampling module 230 provides the de novo protein backbone, i.e., which describes the 3D coordinates of the heavy atoms in each amino acid sequence. The sequence generation model 250 inputs the de novo protein backbone to output the full amino acid sequence that structurally creates the de novo protein backbone. In one or more embodiments, the sequence generation model 250 may further output the DNA sequence for coding the protein. The sequence generation model 250 may be trained as a machine-learning model based on known protein backbones with corresponding known amino acid sequences. In some embodiments, the training module 240 trains the sequence generation model 250 by applying the sequence generation model 250 to a known protein backbone to predict a full amino acid sequence for the known protein backbone. The training module 240 may calculate a loss for each known protein backbone based on a comparison (e.g., a difference) between the predicted full amino acid sequence and the corresponding known amino acid sequence.

The protein folding model 260 validates the full amino acid sequence generated by the sequence generation model 250. The protein folding model 260 inputs an amino acid sequence and determines a protein backbone based on the input amino acid sequence. The protein folding model 260 may be applied to the full amino acid sequence, e.g., generated by the sequence generation model 250, to validate whether the full amino acid sequence successfully folds into the de novo protein backbone. The protein folding model 260 may be retrieved from the third-party database 130. The protein folding model 260 may also be trained by the training module 240, e.g., with known protein backbones and corresponding known amino acid sequences.

The conditioner database 270 stores the one or more baseline conditioner energy components for use by the conditioner module 220. As described above, each type of design condition may be associated with one or more baseline conditioner energy components. The baseline conditioner energy component may include one or more variables to be filled in based on the design condition input by the client device 110. For example, with the symmetry constraint, the conditioner database 270 may store a conditioner energy component for each symmetry.

The protein database 280 stores information on proteins. For example, the protein database 280 stores known protein backbones and corresponding known amino acid sequences, e.g., for training of the various models. The protein database 280 may further store proteins generated by other modules of the analytics system 120. For example, the protein database 280 may store the generated protein backbones and/or the corresponding full amino acid sequences. The protein database 280 may further store results of validation experiments. For example, the analytics system 120 may provide a full amino acid sequence for a de novo protein design to the protein manufacturing system 140 to validate the synthetic protein. The protein manufacturing system 140 may manufacture the synthetic protein and conduct one or more validation experiments to assess characteristics of the manufactured synthetic protein.

### Diffusion Model Training

FIG. 3 illustrates a training process of the diffusion model 210, according to one or more embodiments. The training process may be performed by the analytics system 120, or more specifically the training module 240. The training process is representatively illustrated as the use of three training samples 310, but any number of training samples may be used in the training process. Each reference numeral may be referenced in the singular when referring to individual units or in the plural when referring to the whole set.

To train the diffusion model 210, the analytics system 120 utilizes training samples 310. The training samples 310 may be known protein backbones, e.g., as retrieved from the third-party database 130. The analytics system 120 injects some noise into each known protein backbone to generate a noised state 320 for each known protein backbone. As noted above, the amount of noise may be based on the randomly sampled time step on the time continuum.

The analytics system 120 may filter the training samples 310 to refine the training of the diffusion model 210. For example, the analytics system 120 may deduplicate training samples 310 that are similar. To assess whether two training samples 310 are similar, the analytics system 120 may calculate a distance between the protein backbones of the two training samples 310. If the distance is below a threshold distance, then the analytics system 120 may retain one training sample, whilst excluding the second training sample as redundant. The analytics system 120 may also focus training of the diffusion model 210 to certain types of proteins, e.g., antibodies. In some embodiments, the analytics system 120 may apply a different threshold distance between different types of proteins to bias training of the diffusion model 210 towards particular types of proteins.

In some embodiments, the analytics system 120 may generate multiple training samples 310 from one known protein backbone. For example, the analytics system 120 may generate a first training sample 310 by injecting a first amount of noise to the known protein backbone and may generate a second training sample 310 by injecting a different amount of noise to the known protein backbone. The analytics system 120 may also generate synthetic protein backbones by introducing one or more modifications to the known protein backbones.

The analytics system 120 applies the diffusion model 210 to each noised state 320 to predict a denoised state 330 for each training sample 310. The analytics system 120 calculates a loss 340 for each training sample 310. The loss 340 may be calculated as a difference between the denoised states 330 and the initial states of the training samples 310. The training module 250, thereafter, trains the diffusion model 210 by adjusting parameters of the diffusion model 210 to minimize the losses 340.

### Diffusion Model Deployment for De Novo Protein Generation

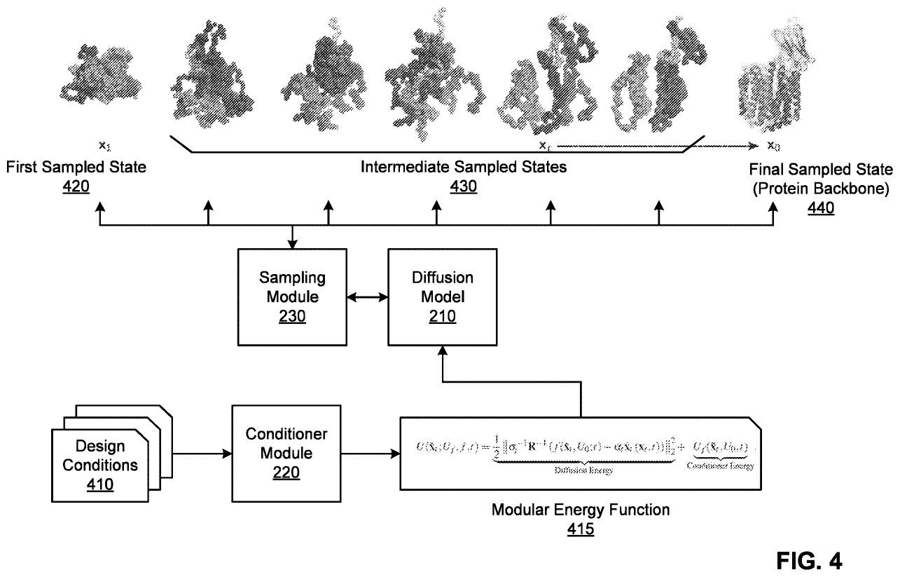

FIG. 4 illustrates deployment of the diffusion model 210 to generate a protein backbone based on a set of one or more design conditions, according to one or more embodiments. The deployment of the diffusion model 210 may be performed by the analytics system 120. The training process is representatively illustrated as performing seven sampling steps, but any number of sampling steps may be used in the deployment process. Each reference numeral may be referenced in the singular when referring to individual units or in the plural when referring to the whole set.

The analytics system 120 receives design conditions 410, e.g., from the client device 110. The design conditions 410 may include one or more restraints, one or more constraints, or some combination thereof. The design conditions 410 specify target characteristics of the protein to be generated. The conditioner module 220 generates a modular energy function 415 based on the design conditions 410. The modular energy function 415 includes a diffusion energy component and one or more conditioner energy components corresponding to the design conditions 410. The modular energy function 415 is utilized by the diffusion model 210 during the sampling process.

The sampling module 230 initializes the sampling process with a first sampled state 420. The first sampled state 420 may be a random state in the multidimensional protein space. In some embodiments, the client device 110 may provide an initial sampled state to serve as a launch point. In a first sampling step, the sampling module 230 inputs the first sampled state 420 into the diffusion model 210 to output a second sampled state (not shown in FIG. 4) based on a gradient of the modular energy function 415. In one or more embodiments, the sampling module 230 may also transform the first sampled state 420 from unconstrained space into constrained space based on the one or more design conditions (e.g., the constraints). The diffusion model 210 denoises in the constrained space. Then the sampling module 230 transforms back into unconstrained space, where the sampling module 230 applies the diffusion model 210 to output the second sampled state. In subsequent sampling steps, the sampling module 230 iteratively inputs a sampled state to output a subsequent sampled state making up the intermediate sampled states 430, e.g., with an incremented reverse time step, trending towards t=0. The final sampled state 440 at t=0 is the de novo protein backbone.

### Diffusion Model Architecture

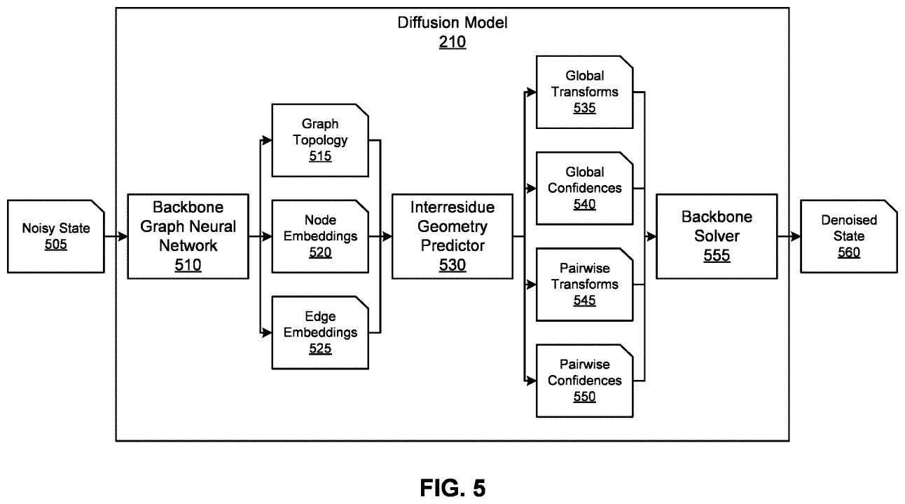

FIG. 5 is a block diagram exampling the architecture of a diffusion model 210, according to one or more embodiments. In FIG. 5, the diffusion model 210 comprises a backbone graph neural network (GNN) 510, an interresidue geometry predictor 530, and a backbone solver 555. In other embodiments, the diffusion model 210 may comprise additional, fewer, or different components than those listed herein. Each reference numeral may be referenced in the singular when referring to individual units or in the plural when referring to the whole set.

The diffusion model 210 is configured to input one noisy state 505 of a protein backbone and to output a denoised state 560 of a protein backbone. The noisy state 505 may be a first sampled state at a time step in the time continuum further towards t=1, whereas the denoised state 560 may be a second sampled state at a time step in the time continuum further towards t=0.

The backbone GNN 510 inputs the noisy state 505 and outputs a graph topology 515, node embeddings 520, and edge embeddings 525. The graph topology 515 describes a graph architecture of the protein backbone. The graph architecture may describe positions of nodes in the graph relative to other nodes and edges between nodes. Each node may have a node embedding 520. Each edge may have an edge embedding 525. The embeddings (e.g., node embeddings 520 and/or edge embeddings 525) may be vector representations (e.g., respectively of nodes and edges in the graph). An edge includes weights that pass values between nodes based on the values of the nodes connected by the edge. The backbone graphical neural network 510 may, in one or more embodiments, a permutation equivariant layer that maps a representation of the graph into an updated representation of the same graph, a local pooling layer that coarsens the graph via downsampling, a global pooling layer that reduces the graph into vector form, or some combination thereof.

The interresidue geometry predictor 530 inputs the graph topology 515, the node embeddings 520, and the edge embeddings 525 to output global transforms 535, global confidences 540, pairwise transforms 545, and pairwise confidences 550. The global transforms 535 indicate a denoised position (e.g., coordinates) of each residue in the protein backbone, while each global transform 535 has an associated global confidence 540 specifying a confidence in the denoised position. The pairwise transforms 545 indicate a denoised relative distance between each pair of residues in the protein backbone, while each pairwise transform 545 has an associated pairwise confidence 550 specifying a confidence in the relative distance.

The backbone solver 555 combines the global transforms 535, the global confidences 540, the pairwise transforms 545, and the pairwise confidences 550 to output the denoised state 560. The backbone solver 555 identifies an optimal solution that attempts to fit all the transforms based on the corresponding confidences. In one or more embodiments, the backbone solver 555 may weight the pairwise transforms 545 more heavily than the global transforms 535. The optimal solution maximizes fitting to transforms with high confidences, e.g., while conversely opting to trade off fitting to transforms with low confidences. The output is the denoised state 560 of the protein backbone, e.g., incrementally denoised from the noisy state 505. In practice, the diffusion model 210 is iteratively applied to incrementally denoise from a first sampled state through a plurality of intermediate sampled states to the final sampled state, i.e., the denoised protein backbone.

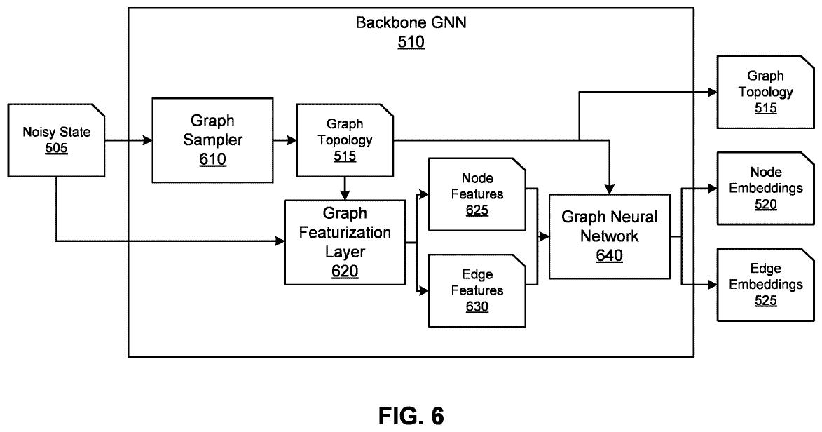

FIG. 6 is a block diagram exampling the architecture of a backbone graph neural network (GNN) 510, according to one or more embodiments. In FIG. 6, the backbone GNN 510 comprises a graph sampler 610, a graph featurization layer 620, and a graph neural network (GNN) 640. In other embodiments, the GNN 510 may comprise additional, fewer, or different components than those listed herein. Each reference numeral may be referenced in the singular when referring to individual units or in the plural when referring to the whole set.

The graph sampler 610 inputs the noisy state 505 to output the graph topology 515. The graph sampler 610 can build the graph topology 515 based on the coordinates of residues in the noisy state 505.

The graph featurization layer 620 inputs the graph topology 515 and the noisy state 505 to output node features 625 and edge features 630. The graph topology 515 defines the graph architecture including number of nodes, position of nodes, and edges connecting pairs of nodes. The node features 625 may encode local geometry, e.g., bond lengths and dihedral angles. The edge features 630 may encode inter-atomic distances, inter-atomic directions, chain distance indicating whether two residues are part of the same polymer chain or different polymer chains, transform features denoting a transform in coordinates of one frame corresponding to one residue to coordinates of the other frame corresponding to the other residue, or some combination thereof.

The GNN 640 inputs the graph topology 515, the node features 625, and the edge features 630 to output the node embeddings 520 and the edge embeddings 525. The GNN 640 is a graph neural network model that is trained to resolve messages passed between nodes according to the edges. The GNN 640 resolves concatenates all messages passed between nodes to generate the node embeddings 520 and the edge embeddings 525.

### Sequence Generation Model Architecture

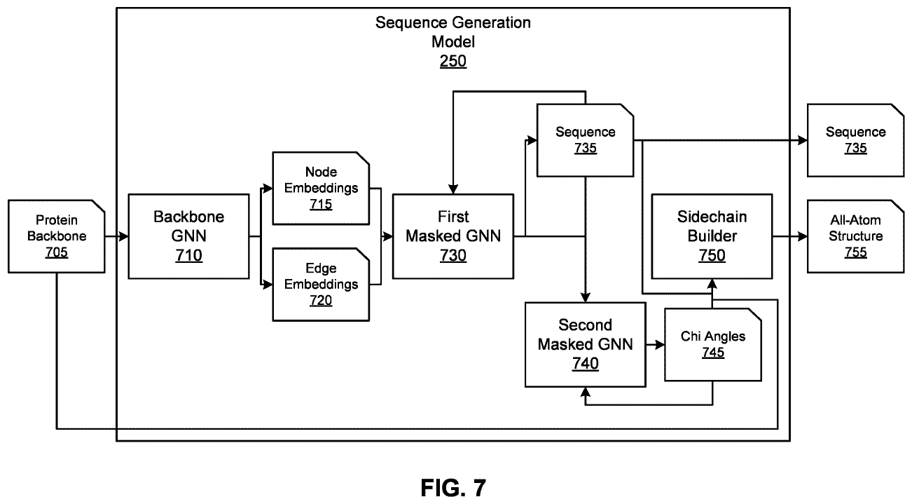

FIG. 7 is a block diagram exampling the architecture of a sequence generation model 250, according to one or more embodiments. The sequence generation model 250 includes a backbone GNN 710, a first masked GNN 730, a second masked GNN 740, and a sidechain builder 750. In other embodiments, the sequence generation model 250 may comprise additional, fewer, or different components than those listed herein. Each reference numeral may be referenced in the singular when referring to individual units or in the plural when referring to the whole set.

The backbone GNN 710 inputs the protein backbone 705 to output node embeddings 715 and edge embeddings 720. In one or more embodiments, the backbone GNN 710 is an embodiment of the backbone GNN 510. The backbone GNN 710 outputs the node embeddings and the edge embeddings which encode features of the nodes and the edges of the protein backbone 705.

The first masked GNN 730 inputs the node embeddings 715 and the edge embeddings 720 to output the sequence 735. The sequence 735 is an amino acid sequence indicating an order and particular amino acid residue in the peptide chain. The first masked GNN 730 may be trained using the known protein backbones and corresponding known amino acid sequences.

The second masked GNN 740 inputs the sequence 735 to determine chi angles 745 of each amino acid residue. Each amino acid is composed of a number of heavy atoms that, due to intermolecular and intramolecular forces, bend at varying angles relative to one another. The chi angles 745 describe the bends in an amino acid residue caused by the forces between the heavy atoms.

The sidechain builder 750 builds amino acid sidechains based on the chi angles 745 and the sequence 735. As noted, each amino acid residue may comprise a sidechain based on heavy atoms present in the amino acid residue. Accordingly, sidechain builder 750 generates the all-atom structure 755 based on the sequence 735 and the chi angles 745. The all-atom sequence 755 comprehensively describes the coordinates of each atom in the protein.

### Methods

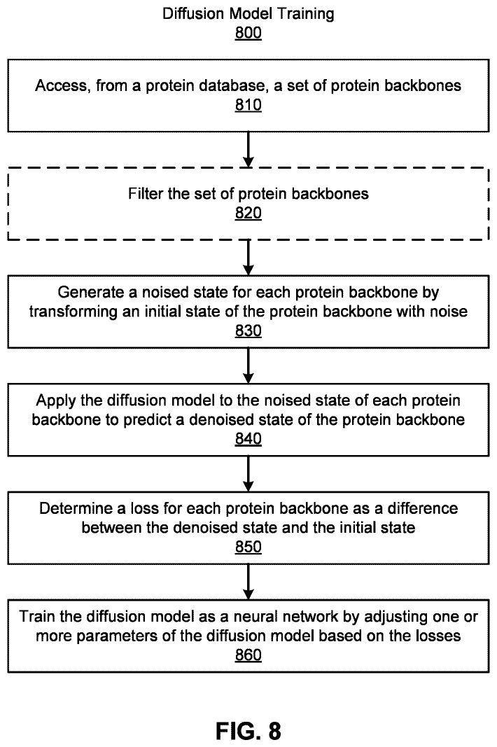

FIG. 8 illustrates a flowchart describing training 800 of a diffusion model for protein design, according to one or more embodiments. The training 800 is described as performed by an analytics system (e.g., the analytics system 120 of FIG. 1). In other embodiments, any step of the training 800 may be performed by another computing device in conjunction with the analytic system. In other embodiments, the training 800 may include additional, fewer, or different steps than those listed herein (as will also be described throughout FIG. 8 description).

The analytics system accesses 810, from a protein database, a set of known protein backbones. The protein database may be a third-party database 130. The protein database stores information relating to known proteins. In some embodiments, the analytics system retrieves the amino acid sequences of the known proteins and generates a protein backbone based on a protein folding model. Each protein backbone describes three-dimensional coordinates of each heavy atom of each amino acid residue in the protein's peptide chain. In other embodiments, the protein backbone may further describe the three-dimensional coordinates of atoms on a sidechain of each amino acid residue.

The analytics system may filter 820 the set of protein backbones to achieve a training set of protein backbones for use in training the diffusion model. In one or more embodiments, filtering includes deduplication of similar protein backbones. To determine if two protein backbones are similar, the analytics system may determine a similarity score as a distance between the coordinates of the two protein backbones. If the similarity score is below a threshold, then the two protein backbones may be deemed sufficiently similar. Accordingly, the analytics system may remove one of the two similar protein backbones. In other embodiments, filtering may include obtaining a high percentage of protein backbones of one or more particular types of protein.

The analytics system generates 830 a noised state for each protein backbone by transforming an initial state of the protein backbone with noise. As the diffusion model learns a reverse-time diffusion process, the analytics system may generate the training samples by simulating forward-time diffusion. The amount of noise added to the initial state may be based on a randomly selected time step on the time continuum. The noise may be Gaussian noise. In some embodiments, the analytics system may generate multiple training samples from one known protein backbone by adding differing amounts of noise to the initial state, thereby generating two distinct noised states.

The analytics system applies 840 the diffusion model to the noised state of each protein backbone to predict a denoised state of the protein backbone. The analytics system applies the diffusion model to predict the initial time step associated with the initial state of the protein backbone. The diffusion model may predict the denoised state based on a gradient of an energy function as applied to the noised state.

The analytics system determines 850 a loss of each protein backbone as a difference between the denoised state and the initial state of the protein backbone. In one or more embodiments, the difference may be a L1 norm, a L2 norm, some other distance calculation, or some combination thereof.

The analytics system trains 860 the diffusion model as a neural network by adjusting one or more parameters of the diffusion model based on the losses. The analytics system may backpropagate through the diffusion model to adjust parameters to minimize the losses between the predicted denoised states and the initial states. In some embodiments, the analytics system may perform batch training of the diffusion model, which generally entails adjusting parameters of the diffusion model to minimizes losses for a batch of training samples. In other embodiments, the analytics system may perform iterative training over epochs. An epoch of training is an instance parameter adjustment from a complete pass of the training set through the diffusion model. The diffusion model may be structured with the architectures described in FIGS. 5 & 6.

The trained diffusion model may be stored in a database of the analytics system, and/or the trained diffusion model may transmitted to one or more other computing devices. When fully trained, the analytics system may deploy the diffusion model in conjunction with a sampling algorithm to generate a de novo protein design.

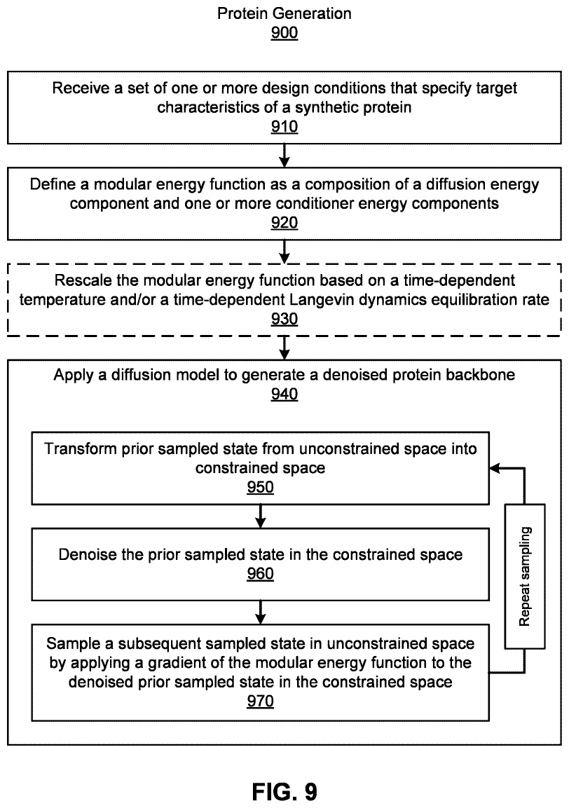

FIG. 9 illustrates a flowchart describing de novo protein generation 900 through deployment of a diffusion model, according to one or more embodiments. The de novo protein generation 900 is described as performed by an analytics system (e.g., the analytics system 120 of FIG. 1). In other embodiments, any step of the de novo protein generation 900 may be performed by another computing device in conjunction with the analytic system. In other embodiments, the de novo protein generation 900 may include additional, fewer, or different steps than those listed herein (as will also be described throughout FIG. 8 description).

The analytics system receives 910 a set of one or more design conditions that specify target characteristics of a synthetic protein. The set of one or more design conditions may be parsed from a text query by a client device. For example, the text query may be “design an antibody with C5 symmetry with Beta hairpin motifs.” The analytics system may parse the text query, e.g., with a NLP model, to determine the one or more design conditions. Following the above example, the analytics system may determine a symmetry constraint of “C5 symmetry,” a text caption restraint of “antibody,” and a secondary structure constraint of “Beta hairpin motif.”

Other types of design conditions may include, but are not limited to, a symmetry constraint, a substructure infilling restraint, a shape constraint, a distance constraint, a substructure root mean squared deviation (RMSD) constraint, a text caption restraint, a sequence constraint, a domain classifier constraint, a secondary structure constraint, etc.

The analytics system defines 920 a modular energy function as a composition of a diffusion energy component and one or more conditioner energy components. The diffusion energy component determines an energy value based on a sampled state of the synthetic protein and a time step of the sampled state. Each conditioner energy component determines an energy value based on the sample state of the synthetic protein and the target characteristic of each design condition. The conditioner energy components may be pulled together based on the set of design conditions received, e.g., from the client device. As such, one design query having one set of design conditions yields a different modular energy function compared to another design query having a distinct set of design conditions. In some embodiments, the conditioner energy components include one or more variables that are filled in based on the received design conditions.

The analytics system may rescale 930 the modular energy function based on a time-dependent temperature and/or a time-dependent Langevin dynamics equilibration rate. The time-dependent temperature enables an adjustable temperature throughout the sampling process, such that the sampling can bias towards high likelihood regions in the multidimension protein space. The time-dependent Langevin dynamics equilibration rate sets the equilibration rate of the Langevin dynamics per unit time. The equilibration rate effectively operates to promote exploration as a counterbalance to low-temperature as a driver of exploitation. The high-likelihood states exhibit increased rates of backbone hydrogen bonding that underlie secondary structure.

The analytics system applies 940 the diffusion model to generate a denoised protein backbone. To generate the denoised protein backbone, the analytics system iteratively samples the multidimensional space with the diffusion model, e.g., trained according to the training 800 in FIG. 8. The initialize the sampling, the analytics system may randomly sample a noised state in the multidimensional protein space.

In one sampling step: the analytics system transforms 950 the prior sample state from unconstrained space into constrained space based on the one or more design conditions. For example, if one design condition is an amino acid sequence constraint, then the analytics system constrains the sampled state to substitute some portion of the sampled state of the protein backbone to include the specified amino acid sequence. In the same sampling step: the analytics system denoises 960 the prior sampled state in the constrained space. The analytic system denoises by determining an amount of noise in the sampled state and removing that amount of noise. In the same sampling step: the analytics system samples 970 a subsequent sampled state in the unconstrained space by applying a gradient of the modular energy function to the denoised prior sampled sate in the constrained space. The subsequent sampled state is one subsequent incremented time step, i.e., towards t→0. The analytics system iteratively performs sampling over a plurality of discrete sampling steps to incrementally progress from the first sampled state to the final sampled state, being the denoised protein backbone.

In further embodiments, the analytics system applies a sequence generation model to the denoised protein backbone to determine a full amino acid sequence for the synthetic protein. The sequence generation model inputs the denoised protein backbone and determines an amino acid sequence that can fold to structurally create the denoised protein backbone. The sequence generation model may further output the sidechain sequences, completing the all-atom structure.

In additional embodiments, the analytics system may perform parallel sampling of the multidimensional protein space with different seed sampled states. The analytics system may use each seed sampled state to generate a diverse set of de novo protein designs that satisfy the set of one or more design conditions. The analytics system may provide the diverse set of de novo protein designs for experimental validation, e.g., of protein folding, of function, etc.

### Conditioner Framework

The previously described restraints and constraints for Langevin dynamics share a common form of implementation: they modify the system coordinates x and/or the total energy U. This suggests a natural building block for a protein programming framework: transformation functions which input and output system states (x, U).

The conditioner framework can be expressed as a function [symbol] : [symbol] ^{N}× [symbol] →Ω⊆ [symbol] ^{M}× [symbol] which maps state-energy pairs in unconstrained input space [symbol] ^{N}× [symbol] to potentially constrained state-energy pairs in Ω⊆ [symbol] ^{M}× [symbol] . For ease of notation, conditioners component-wise [symbol] =(ƒ, U_{ƒ}) in terms of a state update function ƒ: [symbol] ^{N}× [symbol] →Ω_{ƒ}⊆ [symbol] ^{M} and an energy update function U_{ƒ}: [symbol] ^{N}× [symbol] =♯Ω_{U}⊆ [symbol] .

To sample from conditioner-biased diffusion problems, the system uses a gradient-based sampling algorithm, such as Langevin dynamics or Hamiltonian Monte Carlo, on the conditioner-transformed instance of the energy function:

$$U ( {\tilde{x}}_{t} ; {U}_{f} , f , t ) = \frac{1}{2} {‖ {σ}_{t}^{- 1} {R}^{- 1} ( f ( {\tilde{x}}_{t} , {U}_{0} ; t ) - {a}_{t} {\hat{x}}_{t} ( {x}_{t} , t ) ) ‖}_{2}^{2} + {U}_{f} ( {\tilde{x}}_{t} , {U}_{0} ; t )$$

where the gradient V̅_{\hat{x}}U(\tilde{x}ₜ; U_{ƒ}, ƒ, t) for sampling is computed with respect to the unconstrained coordinates \tilde{x}ₜ. These gradients and dynamics can be computed efficiently even for complex composed conditioners by leveraging modern automatic differentiation frameworks.

The Conditioner formulation satisfies the following objectives:
Compositionality. Let [symbol] ₁: [symbol] ^{N}^{₁}× [symbol] →⊆ [symbol] ^{M}^{₁}× [symbol] and [symbol] ₂: [symbol] ^{N}^{₂}× [symbol] →Ω⊆ [symbol] ^{M}^{₂}× [symbol] be Conditioners and assume N₁=M₂⁶. Then [symbol] ₃= [symbol] ₁· [symbol] ₂ is a Conditioner with F₃: [symbol] ^{N}^{₂}× [symbol] →Ω₁⊆ [symbol] ^{M}^{₁}× [symbol] .
Generalized restraints may be realized with state update ƒ(x, U)=x (Identity function) and energy update U_{ƒ}(U, \tilde{x}ₜ, t)=U−log p(y|x, t).
Constraints: Linear Transforms. Distribution-preserving linear transform constraints may be realized with state update ƒ(x, U)=Ax+b and energy update U_{ƒ} (U, \tilde{x}ₜ, t)=U (Identity function).
Constraints: Non-Linear Transforms. Distribution-preserving nonlinear domain constraints may be realized with bijective and differentiable state update ƒ: [symbol] ^{N}× [symbol] →Ω_{ƒ}⊆ [symbol] ^{M} and energy update

$${U}_{f} ( U , {\tilde{x}}_{t} , t ) = U + log det \vert \frac{\partial f}{\partial \tilde{x}} \vert$$

(Change of volume adjustment).
Automated Sampling. Any gradient-based sampling algorithm may be used in concert with the Conditioner-adjusted energy and an annealing schedule on the diffusion time t.

In some embodiments, the modular energy function may condition for sequence and structure. The Conditioner framework is also straightforwardly applicable to joint sampling of sequence and structure, where the joint energy function is defined as:

$$U ( {x}_{t} ; y , t ) = \frac{1}{2} {‖ {σ}_{t}^{- 1} {R}^{- 1} ( f ( {\tilde{x}}_{t} , {U}_{0} ; t ) - {a}_{t} {\hat{x}}_{t} ( {x}_{t} , t ) ) ‖}_{2}^{2} - log p ( {f}_{s} ( {\tilde{s}}_{t} ) \vert {f}_{x} ( {\tilde{x}}_{t} ) , t ) + {U}_{f} ( {\tilde{x}}_{t} , {\tilde{s}}_{t} , {U}_{0} ; t ) .$$

where gradient and dynamics are computed in unconstrained space \tilde{x}ₜ, \tilde{s}ₜ. Discrete Langevin sampling can be implemented in conjunction to sample from sequence space while leveraging gradients for building locally-informed proposals. Sequence and structure gradients can be computed in one pass via automatic differentiation frameworks. Thus, joint sequence and structure sampling can be conditioned on a target objective without needing to train a joint diffusion on sequence and structure at the same time. The valid joint posterior for sequence and structure conditioned on function which may be realized, for example, with a conditional language model for sequence given structure together with a diffusion model for the backbone structure joint marginal.

### Substructure Conditioning

Many protein design tasks including imputation of missing structural data, redesign of an enzyme scaffold given an active site, and redesign of the CDRs of a known antibody framework require exact specification of the known structural coordinates. In this section, a method is disclosed that allows for such specification as a hard constraint on the reverse diffusion trajectories.

Substructural conditioning can bias sampling by adding a conditional score term V̅ₓ log pₜ(y|x) to the drift component in the reverse SDE. To enforce y in these regimes one must upweight the conditional score relative to the prior score function which can result in a reduction in the likelihood (or ELBO) of the samples drawn, or even in numerical instability.

The method presented below leverages an approach where the equilibrium states of a system are sampled by simulating the dynamics of an auxiliary system with a modified mass matrix. If the mass matrix is chosen appropriately, the original system's configuration space can be sampled more efficiently.

The method works by initializing x₁ in a way that enforces condition y, so that p₁(y|x₁)=1, and then integrating a modified Annealed Langevin Dynamics SDE backwards in time to sample from p₀(x|x₁), where the dynamics are modified to be y preserving by using a mass matrix that assigns higher mass to particles closer (in chain distance) to known coordinates and assigning infinite mass to known atoms. Samples drawn using this method satisfy y with probability 1.

Let S, M⊂[1, . . . , N] denote the atoms comprising the unknown scaffold and known motif, respectively, throughout this section.

It is known that for x~ [symbol] (μ, Σ). The system can partition the coordinates as above into subsets M, S and write:

$$x = [ \begin{matrix}{x}^{S} \\ {x}^{M}\end{matrix} ] with μ = [ \begin{matrix}{μ}^{S} \\ {μ}^{M}\end{matrix} ] and \sum = [ \begin{matrix}{\sum }_{S S} & {\sum }_{S M} \\ {\sum }_{M S} & {\sum }_{M M}\end{matrix} ]$$

that (x^{s}|x^{M}=a)~ [symbol] (μ̅,Σ̅) where:

$$\overline{μ} = {μ}^{S} + {\sum }_{S M} {\sum }_{MM}^{- 1} ( a - {μ}^{M} )$$

$$and :$$

$$\overline{\sum } = {\sum }_{S S} - {\sum }_{S M} {\sum }_{MM}^{- 1} {\sum }_{M S}$$

where inverse matrices are understood to denote pseudo-inverses. The system also computes the Cholesky factorization R̅R̅^{T}=Σ̅.

To draw an approximate conditional sample from

$$p ( {x}_{0}^{S} \vert {x}_{0}^{M} = a ) ,$$

the system proceeds as follows: first, the system samples

$${x}_{1}^{s} \sim 𝒩 ( \overline{μ} , \overline{Σ} )$$

the conditional prior, set

$${x}_{0}^{M} = a ,$$

and integrate a modified Annealed Langevin Dynamics SDE:

$$dx = - \frac{{β}_{t} Ψ}{2} {λ}_{0} {RR}^{T} {∇}_{x} log {p}_{t} ( x ) dt + \sqrt{{β}_{t} Ψ} R d \overline{w}$$

backwards in time, where the matrices are R̅, R̅^{T} are broadcast to the correct size with the conditioned on rows and columns filled by zeroes.

In additional embodiments, the system incorporates a reconstruction-guidance based score term. While this can introduce some instability to the sampling it can sometimes improve sample quality. To do so, in the energy block formulation the system defines:

$${U}_{f} ( {x}_{t} , U , t ) = U + {‖ {{\hat{x}}_{θ} ( {x}_{t} , t )}^{M} - {{x}_{t}}^{M} ‖}_{2}^{2}$$

$$where$$

$${x}_{t} = f ( {\tilde{x}}_{t} ) = \overline{R} {\overline{R}}^{- 1} {x}_{t} + \overline{μ} .$$

### Distance-Based Constraints

In one or more embodiments, a distance constraint specifies that one or more specific residue pairs be in spatial proximity (i.e., form a “contact”). Such a conditioner could be used, for example, in designing binders, to ensure that the desired binding site is being engaged. Or it could be used to insure some desired topological properties—i.e., the proximity of N- and C-termini (e.g., for ease of circular permutation).

To condition on a contact between atoms i and j, the system is seeking the probability that the distance between these two atoms in the fully denoised structure is below some desired cutoff c,

$${d}_{0}^{ij} < c ,$$

given a noised sample at time t and the corresponding distance

$${d}_{t}^{ij} .$$

In one or more embodiments, the system trains a time-dependent classifier pₜ(y|x(t)) to classify noisy inputs. For the case of a contact classifier, the system can directly compute the desired probability analytically. By definition of the forward noise process, the i-th coordinate of the protein at time 0 and t are related to each other by:

$${x}_{0}^{( i )} = \frac{{x}_{t}^{( i )}}{{α}_{t}} - {\sqrt{( 1 - {{α}_{t}}^{2} )} [ Rz ]}_{i}$$

Below are the derivations of the distribution

$${d}_{0}^{ij}$$

cases of Brownian and globular noise schedules.

Here:

$${[ Rz ]}_{i} = γ {\sum }_{k}^{i} {𝓏}_{k} - \frac{γ}{N} {\sum }_{j = 1}^{N} {\sum }_{k = 1}^{j} {𝓏}_{k} + {δ𝓏}_{1}$$

and therefore:

$${x}_{0}^{( j )} - {x}_{0}^{( i )} = \frac{{x}_{t}^{( j )} - {x}_{t}^{( i )}}{{α}_{t}} - γ \sqrt{( 1 - {{α}_{t}}^{2} )} {\sum }_{k = j}^{i} {𝓏}_{k}$$

### But as

$${\sum }_{k = j}^{i} {𝓏}_{k} \sim 𝒩 ( 0 , \vert i - j \vert )$$

independence of {zᵢ}, the system has

$${x}_{0}^{( j )} - {x}_{0}^{( i )} \sim 𝒩 ( \frac{{x}_{t}^{( j )} - {x}_{t}^{( i )}}{{α}_{t}} {γ}^{2} ( 1 - {{α}_{t}}^{2} ) \cdot \vert i - j \vert ) ,$$

so that:

$$\frac{{( {d}_{0}^{ij} )}^{2}}{\vert i - j \vert ( 1 - {{α}_{t}}^{2} ) {γ}^{2}} \sim NonCenteralChiSquared [ \frac{{( {d}_{t}^{ij} )}^{2}}{\vert i - j \vert ( 1 - {{α}_{t}}^{2} ) {α}_{t} {γ}^{2}} , k = 3 ]$$

For a contact threshold c>1, the system has:

$${d}_{0}^{ij} < c ⇔ ( {d}_{0}^{ij} ) < {c}^{2} ⇔ \frac{{( {d}_{0}^{ij} )}^{2}}{\vert i - j \vert ( 1 - {{α}_{t}}^{2} ) {γ}^{2}} < \frac{{c}^{2}}{\vert i - j \vert ( 1 - {{α}_{t}}^{2} ) {γ}^{2}}$$

and so

$${p}_{t} ( {d}_{0}^{ij} < c \vert {X}_{T} )$$

is given exactly by the cumulative density function of the noncentral chi-squared distribution above, evaluated at c²[|i−j|(1−αₜ²)γ²]⁻¹.

For the globular chain noise process, the system instead utilizes:

$${[ Rz ]}_{i} = a {\sum }_{k = 2}^{i} {b}^{i - k} {𝓏}_{k} + a \frac{{b}^{i - 1}}{\sqrt{1 - {b}^{2}}} {𝓏}_{1}$$

By substituting:

$${x}_{0}^{( j )} - {x}_{0}^{( i )} = \frac{{x}_{t}^{( j )} - {x}_{t}^{( i )}}{{α}_{t}} + \sqrt{1 - {α}_{t}^{2}} ( {[ Rz ]}_{j} {- [ Rz ]}_{i} ) .$$

So that

$${x}_{0}^{( j )} - {x}_{0}^{( i )} ∼ 𝒩 ( \frac{{x}_{t}^{( j )} - {x}_{t}^{( i )}}{{α}_{t}} , ( 1 - {α}_{t}^{2} ) Var ( {[ Rz ]}_{j} {- [ Rz ]}_{i} ) ) .$$

But assuming j>i:

$$Var ( {[ Rz ]}_{j} {- [ Rz ]}_{i} ) = \frac{2 {a}^{2} ( 1 - {b}^{j - i} )}{1 - {b}^{2}} = : {σ}_{j - i}^{2} .$$

It then follows that:

$$\frac{{x}_{0}^{( j )} - {x}_{0}^{( i )}}{\sqrt{1 - {α}_{t}^{2}} {σ}_{j - i}} ∼ 𝒩 ( \frac{{x}_{t}^{( j )} - {x}_{t}^{( i )}}{{σ}_{j - i} {α}_{t} \sqrt{1 - {α}_{t}^{2}}} , I )$$

and finally:

$$\frac{{( {d}_{0}^{ij} )}^{2}}{( 1 - {α}_{t}^{2} ) {σ}_{j - i}^{2}} ∼ NonCentralChiSquared [ \frac{{( {d}_{t}^{ij} )}^{2}}{{σ}_{j - i}^{2} {α}_{t} ( 1 - {α}_{t}^{2} )} , k = 3 ] .$$

### Sub-Structure RMSD

In one or more embodiments, a design condition of sub-structure RMSD may specify a particular structural motif to include in the protein backbone. This motif can be an arbitrary substructure, composed of any number of disjoint backbone segments, that should be present in the final generated structure. In practice, such a motif could represent a functional (e.g., catalytic) constellation of residues or a metal/small-molecule binding site—this could be useful for designing enzymes or other functional proteins, by exploring ideas around a core functional mechanism. In another example, the motif could correspond to a scaffolding part of the molecule that may be important to preserve, e.g., the binding scaffold that can admit different loop conformations. Or the motif could represent a desired epitope that to present on the surface of a generated protein in the context of vaccine design.

The task of determining whether the pre-specified motif is present in a given structure S is simple, the system can, for example, find the substructure of S with the lowest optimal superposition root-mean-squared-deviation (RMSD) to the motif in question and ask whether this RMSD value is below a desired cutoff. But in the diffusion model, the system needs to determine the probability that the desired motif is expressed in a noisy structure at the current time point in the diffusion.

Specifically, if xₜ∈ [symbol] ^{N×3} is the coordinate array and the forward diffusion process is represented by:

$${x}_{t} = {α}_{t} {x}_{0} + \sqrt{1 - {α}_{t}^{2}} R ϵ , ϵ ∼ N ( 0 , I )$$

then the system aims to express pₜ(y|xₜ) the probability that x₀ contains the motif given xₜ, where y stands for the condition of motif presence (e.g., as defined by RMSD to a template motif below a desired cutoff). If the presence of a motif is defined in terms of optimal-alignment best-fit RMSD being below a cutoff, the system aims to understand how this RMSD behaves (in a probabilistic sense) as a function of noise. Further, as it is not given where within xₜ the motif may be (i.e., the system would not know a priori the matching between motif atoms and a sub-structure of the target structure), pₜ(y|xₜ) needs to integrate information for the full structure xₜ to determine possible motif location(s). Achieving this analytically seems non-trivial. For this reason, here the system considers an empirical approach to expressing pₜ(y|xₜ).

The goal is to observe the behavior of optimal-alignment best-fit RMSD in practice, as a function of αₜ, using a set of reasonable structures and diverse motifs, and find an analytical approximation for its probability distribution. Specifically, given a motif m and a structure represented by xₜ, lerₜt represent the RMSD of optimal alignment of m onto xₜ (i.e., the lowest RMSD between atoms of m and any sub-structure of xₜ, and r₀ represents the RMSD induced by the same matching in the context of structure x₀, and r₀. The system seeks to approximate the cumulative distribution function F(r₀−rₜ|xₜ, at).

With this, the system can calculate pₜ(y|xₜ) as:

$${p}_{t} ( y \vert {x}_{t} ) = p ( {r}_{0} < σ \vert {x}_{t} ) = p ( {r}_{0} - {r}_{t} < σ - {r}_{t} \vert {x}_{t} ) = F ( σ - {r}_{t} \vert {x}_{t} , {α}_{t} ) ,$$

where σ is the desired RMSD cutoff for classifying the existence of the motif.

Clearly, the distribution of rt (and Δrₜ=r₀−rₜ) should depend on αₜ. But these distributions should also depend on the size and complexity of the motif. For example, in the extreme case when the motif consists of a single atom, rₜ will always be zero. On the other hand, for large and complex motifs, the system may expect rₜ to increase rapidly with added noise.

The simplest surrogate for motif complexity is its size—i.e., the number of residues it involves. However, under the noise model, the atoms closer to each other in the protein chain will move in a more correlated manner than those that are farther apart. So it should matter whether the motif consists of multiple short disjoint segments matching to far-away (in sequence) portions of the target structure versus a motif consisting of one long contiguous segment. As a purely empirical measure to capture this notion, the system utilizes the following effective length definition:

$${L}_{e} = - log [ \frac{2}{n ( n - 1 )} {\sum }_{i = 1}^{N - 1} {\sum }_{j = i + 1}^{N} \frac{1}{\sqrt{\vert i - j \vert}} C ( i , j ) ]$$

where C(i, j) is an indicator function that is 1 if atoms i and j are part of the same chain and 0 otherwise. The motivation for the inverse square root of the index distance is from Brownian motion (displacement distance growing as the square root of time, here the number of atom hops). And the motivation for ignoring atom pairs from different chains is that these move independently under the noise model. In practice, Lₑ appears to better explain variation of rₜ−r₀ than just pure number of motif residues L, despite the fact that overall Lₑ correlates somewhat closely with log (L).

The distribution of r₀−rₜ depends on αₜ and Lₑ. To get a sense of the general shape of this distribution and its dependence on αₜ, the system can take slices of the training data with at in different narrow ranges. Inspection and fitting of these αₜ-window histograms of r₀−rₜ suggested that the Gumbel family of distribution should work reasonably well for describing the observed variations.

The dependence on Lₑ can be captured defining the parameters of the Gumbel distribution as functions of Lₑ. Towards defining a reasonable functional form, the system consider extremes. The Gumbel distribution has two parameters-location μ and scale β. The latter is solely responsible for the variance

$$( i . e . , \frac{{π}^{2}}{6} {β}^{2} )$$

and the mean is contributed to by both (μ+βγ), where γ is the Euler-Mascheroni constant, or approximately 0.577). For a motif that only has one atom, Δrₜ is a delta function at 0, meaning that both μ and β would be zero. And in general, for small (and simple) motifs the system would expect μ and β to be low, while for large (and complex) motifs the system would expect it to be high. Thus, both μ and β should be monotonically increasing functions of Lₑ that pass through the origin. Experimentation with different curve families under these criteria, using the overall data likelihood as the objective metric (see below), the system arrived at the simple linear parameterization option as being best, i.e., where μ=μₛLₑ and β=βₛLₑ with us and Bs being fitting parameters.

With the parameterization choices above, the fitting approach employs the following steps.

For 50 equally-spaced at windows, fit the observed Δrₜ=rₜ−r₀ to Gumbel distributions, whose location and scale parameters linearly depend on Lₑ of each motif, using likelihood maximization. Specifically, the likelihood function being maximized was:

$$log ℒ = {\sum }_{i = 1}^{{N}_{D}} - log ( {β}_{s} {L}_{e}^{i} ) - \frac{Δ {r}_{t}^{i} - {μ}_{s} {L}_{e}}{{β}_{s} {L}_{e}} - exp ( - \frac{Δ {r}_{t}^{i} - {μ}_{s} {L}_{e}}{{β}_{s} {L}_{e}} )$$

$$where {L}_{e}^{i} and Δ {r}_{t}^{i}$$

are the effective motif length and Δrₜ is the i-th data point, respectively, and N_{D} is the number of data points. The result of this procedure then estimates μₛ and βₛ parameters specific for the current at window.

Next, the system fits the parameters μₛ and βₛ as functions of at analytically. The functional form chosen for both parameters was k·(1−αₜ²)ⁿ, such that at αₜ=1 both parameters become zero (i.e., as the noise level reaches zero, the Δr distribution should approach a delta function).

### Symmetry Constraint

Built from identical subunit proteins, many protein complexes are assembled symmetrically. Many symmetric complexes such as tube-shaped channel proteins and icosahedral viral capsids are biologically important. Incorporating symmetry in computational protein generation holds promise in designing large functionalized protein complexes. To fully explore the sampling of protein complexes subject to symmetry constraints, the system symmetrizes the underlying ODE/SDE sampling to satisfy any prescribed Euclidean symmetries. Incorporating group equivariance in machine learning has been an important topic in the machine learning community. Incorporating space group symmetries is critical in molecular simulations.

Let

$$G = {[ g ]}_{i = 0}^{N}$$

be a collection of symmetry operations that form a group such as point groups and space groups. For point sets in [symbol] ³, these symmetry operations can be represented as a set of orthogonal transformations (rotation/reflection) and translations. For synthesizing symmetric protein complexes, the system want to sample complexes xₜ∈ [symbol] ^{N×n×3} which are built from N=|G| identical single-chain proteins x E [symbol] ^{M×3} where M is the number of residues for each subunit. The SDE solving process produces final sample with:

$${x}_{0} = sde\_solve ( {x}_{T} )$$

For sample generation to respect symmetries for an arbitrary group G, the SDE/ODE dynamics need to be G-invariant up to a permutation of subunits. Let · represent the symmetric operations (rotation, reflection, and translation) performed on point sets in [symbol] ³, the system define the sampling procedure sde_solve: [symbol] ^{|G|×n×3}→ [symbol] ^{|G|×n×3} with x₀=sde_solve(x_{T}) being the desired samples. The sampling procedure needs to follow the following invariance condition:

$$sde\_solve ( {x}_{T} ) = gsde\_solve ( {x}_{T} ) = σ ( g ) sde\_solve ( {x}_{T} ) , ∀ g ∈ G$$

where gᵢ indicates the i-th group element in G and the system impose an arbitrary order on G and the method is equivariant to the permutation of subunits. σ(g) is the induced permutation operation satisfying the relation: gG=σ(g)G, as computed from the group multiplication table (also called the Caley table).

The first equality is trivially satisfied if ƒ(⋅) or the underlying gradient update is E(3) equivariant, as G consists of only orthogonal transformations and translations. However, the second equality is generally not satisfied. For molecular simulations where the Hamiltonian dynamics is used, the second equality can be satisfied if (i) the energy function is E(3) invariant, and (ii) the initial x^{T} and

$$\frac{{dx}_{T}}{dt}$$

are symmetric, i.e., gᵢ·x_{T}=σ(g),

$${g}_{i} \cdot \frac{d {x}_{T}}{d t} = σ ( g ) \frac{d {x}_{T}}{d t} .$$

At each successive time step, x_{T} automatically satisfies the prescribed G-symmetry. This approach confines both the position and momentum update to ensure the sampled configurations remain symmetric.

However, this is not the case with SDE/ODE sampling in the framework. There are, in some embodiments, three origins of symmetry-breaking error. (i) ƒ(x_{T}, t) uses distances as features and is automatically E(3) equivariant. However, because the protein feature graphs are generated probabilistically, ƒ(gx_{T},t)≠gf(x_{T}, t) with each subunit protein having different geometric graphs, although they are symmetric. (ii) The polymer structured noise is randomly sampled from [symbol] (x_{T}; μ, Σ), so each subunit protein has different chain noises. (iii) The sampling procedure requires solving an ODE/SDE which is vulnerable to accumulated integration error. Integration error can induce unwanted geometric drifts such as rotation and translation, and be a substantial symmetry breaking force.

In one or more embodiments, the system employs the symmetric sampling approach as a constrained transformation formalism implemented as a conditioner block. Using the representations of G roto-translations of G, the system demonstrate the building of protein symmetric assemblies from an asymmetric unit (AU) chain \tilde{x} through symmetrization. The system commences with the mathematical formulation of the transformation, subsequently elucidating the induced linear transformation on the intrinsic gradient dynamics. Representing G as N×n×3, a collection of rotation matrices G, the system define the constrained transformation as:

$${x}_{T} = f ( {\tilde{x}}_{T} , t ) = symmetrize ( {\tilde{x}}_{t} ) = G {\tilde{x}}_{t}$$

with the equivalent indexed multiplication as:

$${[ {x}_{t} ]}_{n i m} = {\sum }_{j} {{G}_{n i j} [ {\tilde{x}}_{t} {x}_{t} ]}_{m j}$$

where n is the index of group elements, m is the index for atoms in AU, and i, j are Euclidean indices. The associated diffusion energy transformation is:

$$\frac{1}{\vert G \vert} {U}_{f} ( {x}_{t} ) = \frac{1}{\vert G \vert} {‖ {σ}_{t}^{- 1} {R}^{- 1} ( {x}_{t} - {α}_{t} {\hat{x}}_{t} ) ‖}_{2}^{2}$$

The energy is averaged with |G| to account for the diffusion energy in individual AU with M atoms, the system can compute the Jacobian of the transformation ƒ: [symbol] ^{M×3}→ [symbol] ^{N×M×3}.

$$\frac{df ( {x}_{t} )}{d {\tilde{x}}_{t}} = G \to {[ \frac{{d [ f ( {x}_{t} ) ]}_{n i m}}{d {\tilde{x}}_{t}} ]}_{j m} = {G}_{n i j}$$

To derive the transformed dynamics, the system incorporates a one-solver step for the reverse Langevin dynamics (the analysis is the same for reverse diffusion):

$${\tilde{x}}_{t + d t} = {\tilde{x}}_{t} - \frac{1}{2} R {{R}^{T} [ \frac{df ( {\tilde{x}}_{t} )}{d {\tilde{x}}_{t}} ]}^{T} [ \frac{dU ( {x}_{t} )}{{dx}_{t}} ] dt + R d \overline{w}$$

The system analyzes the induced gradient transform with its associated indexed representation:

$$\frac{{dU}_{f} ( {x}_{t} )}{d {\vec{x}}_{t}} = {[ \frac{df ( {\tilde{x}}_{t} )}{d {\vec{x}}_{t}} ]}^{T} [ \frac{{dU}_{f} ( {x}_{t} )}{{dx}_{t}} ] = {G}^{T} \frac{{dU}_{f} ( {x}_{t} )}{{dx}_{t}}$$

$$\frac{{dU}_{f} ( {x}_{t} )}{{d [ {\tilde{x}}_{t} ]}_{j m}} = {\sum }_{n} {\sum }_{i} {{G}_{n i j} [ \frac{{dU}_{f} ( {x}_{t} )}{{dx}_{t}} ]}_{nim} .$$

Observe that in the gradient transformation, summation occurs over indices i, contrasting with index j used in the forward transformation. This method inherently pulls gradients back to AU. The computation of the transformed gradient can be adeptly handled using auto-differentiation, specifically as vector-Jacobian products. Additionally, the gradient accumulated onto AU are also averaged by the number of chains in the tessellated domain by dividing the gradient with |G|.

The system next analyzes the transformed solver step with the pull-back gradient transform:

$$f ( {\tilde{x}}_{t} + d {\tilde{x}}_{t} ) = f ( {\tilde{x}}_{t} - \frac{1}{2} R {{R}^{T} [ \frac{df ( {\tilde{x}}_{t} )}{d {\tilde{x}}_{t}} ]}^{T} \frac{1}{\vert G \vert} [ \frac{dU ( {x}_{t} )}{{dx}_{t}} ] d t + R d \overline{w} ) = G ( {\tilde{x}}_{t} - \frac{1}{2} R {R}^{T} {G}^{- 1} \frac{1}{\vert G \vert} [ \frac{dU ( {x}_{t} )}{{dx}_{t}} ] dt + Rd \overline{w} )$$

where G is the symmetrize component and

$$( {\tilde{x}}_{t} - \frac{1}{2} R {R}^{T} {G}^{- 1} \frac{1}{\vert G \vert} [ \frac{dU ( {x}_{t} )}{{dx}_{t}} ] d t + R d \overline{w} )$$

is the folding to AU component.

The constrained transformation has a nice interpretation, the solver step first folds the infinitesimal change back followed by symmetrization. Another option to pull the gradients is perform a “broadcasting” operation from a single AU (indexed with u) of x. This is also a valid gradient transformation that ensures G-invariance.

$$f ( {\tilde{x}}_{t} + d {\tilde{x}}_{t} ) = f ( {\tilde{x}}_{t} - \frac{1}{2} R {{{R}^{T} [ G ]}_{u}^{- 1} [ \frac{d U ( {x}_{t} )}{d {x}_{t}} ]}_{u} d t + R d \overline{w} ) .$$

For efficient memory sampling of large symmetric assemblies, the system may reduce the number of chains using chain subsampling techniques. This approach allows us to concentrate on updating a specific subset, denoted as S⊂[1, . . . , |G|], of subunits in x_{T}, thereby conserving both memory and computational time.

Given a designated subunit i, the subset S is derived by selecting the k-nearest neighbor (k-NN) subunits. This selection is determined by the distances between the geometric centers of the subunits, ensuring the incorporation of short-range interactions between them. Through this method, K subunits are chosen, where K represents the count of neighbors the denoiser interacts with during each integration phase.

This randomized selection not only ensures that the gradient update remains globally consistent, but also prevents potential structural clashes and suboptimal contact formations.

This procedure, at its core an index selection mechanism, can also be depicted as a linear transformation using a sparse matrix comprised of 0s and 1s. By harnessing inter-chain distances, the system are equipped to select K<|G| chains following an exhaustive symmetric tessellation. This method of subsampling aligns with established techniques in molecular simulations that employ periodic boundary conditions. To further understand the subsampling process, it is interesting to note that, much like the tessellation method, the subsampling can be described as:

$${x}_{t} = f ( {\tilde{x}}_{t} , t ) = {\tilde{x}}_{t}^{S} = subsample ( {\tilde{x}}_{t} ) = S {\tilde{x}}_{t}$$

$$\frac{df ( {x}_{t} )}{d {\vec{x}}_{t}} = S ∈ {[ 0 , 1 ]}^{[ G \vert N ] \times M}$$

where S is the chain selection matrix of size (NK×M) where K<N is the number of chains selected for computation, and this can be efficient.

The conditioner block formalism provides the flexibility to seamlessly incorporate restraint energy during energy updates. To ensure optimal contact and packing, the system integrates an R_{g} penalty through an inter-chain potential or flat-bottom potential. This serves to maintain both the inter-chain distance and the Asymmetric Unit (AU) Radius of Gyration as follows:

$${U}_{f} ( {x}_{t} , U , t ) = U + {U}_{{R}_{g}} ( {x}_{t} ) = U + {‖ {R}_{g} ( {x}_{t} , t ) - 〈 {R}_{g} 〉 ‖}_{2}^{2}$$

The proposed samplers can also be combined with other conditioners (substructure, natural language, shape, etc.) to realize symmetric assembly design with controllable functions.

Putting together, the composed transformation is:

$$x = subsample ( symmetrize ( \tilde{x} ) )$$

$${U}_{f} ( x , U , t ) = U + {U}_{{R}_{g}} ( X ) + {U}_{{R}_{g}} ( subsample ( symmetrize ( \vec{x} ) ) )$$

The system may further condition on other point groups including Cn (cyclic symmetry), Dn (dihedral symmetry), T (tetrahedral symmetry), O (octahedral symmetry), I (icosahedral symmetry). For all the samples, the inverse temperature was set to 8 and the equilibration rate to 8 with the Heun SDE solver that integrates from 1 to 0 for 400 steps. Subunit k-NN sampling with K=6. When K>|G|, K was set to equal |G|.

### Shape Conditioning

Proteins often realize particular functions through particular shapes, and consequently being able to sample proteins subject to generic shape constraints would seem to be an important tool for fully realizing the potential of protein design. Pores allow molecules to pass through biological membranes via a doughnut shape, scaffolding proteins spatially organize molecular events across the cell with precise spacing and interlocking assemblies, and receptors on the surfaces of cells interact with the surrounding world through precise geometries. Here methods are introduced to explore and test generalized tools for conditioning on volumetric shape specifications.

The shape conditioning approach is based on Optimal Transport, which provides tools for identifying correspondences and geometric distances between objects, such as the atoms in a protein backbone and a point cloud sampled from a target shape. The system leverages two metrics from the optimal transport theory: (i) the Wasserstein distance which can measure the correspondence between point clouds in absolute 3D space, and (ii) the Gromov-Wasserstein distance, which can measure the correspondences between objects in different domains by comparing their intra-domain distances or dissimilarities. Because it leverages relational comparisons, Gromov-Wasserstein can measure correspondences between unaligned objects with different structures and dimensionalities such as a skeleton graph and a 3D surface or even between unsupervised word embeddings in two different languages.

When adding heuristic gradients to the diffusion based on just the Wasserstein distance, there is huge degeneracy in potential volume-filling conformations would often lead to jammed or high contact-order solutions. The system accelerates convergence by breaking this degeneracy with a very coarse “space-filling plan” for how the fold should map into the target point cloud, which the prior can then realize with a specific protein backbone.

The system can leverage Gromov-Wasserstein (GW) optimal transport. The system (i) generates an idealized distance matrix for a protein based on the scaling law Dᵢⱼ=7.21×|i−j|, (ii) computes the distance matrix for the target shape, and (iii) solves for the Gromov-Wasserstein optimal transport given these two distance matrices yielding a coupling matrix K_{GromovWasserstein} with dimensionality Nₐₜₒₘₛ× Nₚₒᵢₙₜₛ. This coupling map sums to unity and captures the correspondence between each atom in the abstract protein chain and each point in the target point cloud. The system may incorporate a small amount of entropy regularization to solve the optimal transport problem.

In the inner loop of sampling, the system can combine the Gromov-Wasserstein coupling with simple Wasserstein couplings as a form of regularization towards the fold “plan”. The final loss is then:

$$ShapeLoss ( x , r ) = {\sum }_{i , j} ( {K}_{i j}^{G W} + {K}_{i j}^{w} ( x , r ) ) ‖ {x}_{i} - {r}_{j} ‖$$

where the system computes the Wasserstein optimal couplings

$${K}_{i j}^{W}$$

with the Sinkhorn algorithm. This yields a fast, differentiable loss that can be used directly for sampling.

The system weights the ShapeLoss(x, r) term with the scaling factor:

$${w}_{t}^{( shape )} = Clamp ( \sqrt{{SNR}_{t}} , [ 0.001 , 3. ] )$$

and then add its gradient directly to the loss during sampling. So the weighted objective is:

$${ShapeLoss}_{t} ( x , r ) = Clamp ( \sqrt{{SNR}_{t}} , [ 0.001 , 3. ] ) {\sum }_{i , j} ( {K}_{ij}^{GW} + {K}_{ij}^{W} ( x , r ) ) ‖ {x}_{i} - {r}_{j} ‖$$

The system successfully rendered letters and numbers from the English alphabet in the Liberation Sans font, extruded these 2D images into 3D volumes, and then sampled isotropic point clouds from these volumes.

### Residue, Domain, and Complex-Level Classification

Noised backbone coordinates obtained from the PDB are passed as input to the model, along with a scalar 0<t<1 denoting the time during diffusion (indexed between zero and one) that the noise was sampled at. The model optionally can consume sequence information if available.

The time component is encoded with a random Fourier featurization. The provided sequence is encoded with a learnable embedding layer of amino acid identity. Backbone coordinates are passed to our ProteinFeatureGraph that extracts 2-mer and chain-based distances and orientations. These components are summed and passed to the neural network.

The encoder is a message-passing neural network. The graph is formed by taking K=20 nearest neighbors and sampling additional neighbors from a distribution according to a random exponential method.

Node and edge embeddings are passed to each layer, with each node being updated by a scaled sum of messages passed from neighbors. The message passed from node i to node j is obtained by stacking the embeddings at node i, those at node j, and E, and passing these to a multi-layer perceptron (e.g., implemented with one or more hidden layers). Edges are updated similarly. Each layer also applies layer normalization (along the channel dimension) and dropout (dropout probability=0.1). After processing by the MPNN, node embeddings are passed to a different classification head for each label. If a head corresponds to a chain-level label, residues from each chain are pooled using an attentional pooling layer. The resulting embeddings are then passed to an MLP with 1 hidden layer to output logits for each label.

The model may be trained to predict the following labels: CATH, PFAM, Funfam, Organism, Secondary Structure, Interfacial Residue. The loss for predicting each label is quantified using cross entropy loss, and all components are summed and weighted equally.

The model may be trained for 50 epochs with an Adam optimizer with default momentum settings (betas=(0.9,0.999)), the learning rate is linearly annealed from 0 up to 0.0001 over the first 10,000 steps then kept constant. During training, first a time stamp 0<t<1 is sampled uniformly, then noise is sampled from the globular covariance distribution, injected into the backbone coordinates, and fed to the model. Next, label predictions are made, loss are computed, and parameters are updated with the Adam optimizer.

In one or more implementations, the classification model has 4 layers, the size of node feature dimension is 512 and the edge feature dimension is 192, node update MLP has hidden dimension 256 with 2 hidden layers, and edge update MLP has hidden dimension 128 with 2 hidden layers.

### Natural Language Annotations

Recent advances in text-to-image diffusion models have produced qualitatively impressive results using a natural language interface. Given the open availability of pre-trained language models and a corpus of protein captions form large scientific databases such as the PDB and UniProt, the system implements a natural language interface to protein backbone generation.

To do so, the system uses a protein captioning model (The protein caption model), which predicts p(y|xₜ), where y is a text description of a protein and xₜ is a noised protein backbone. This conditional model, when used in conjunction with the structural diffusion model presented in the main text, can be used as a text-to-protein backbone generative model.

To build a caption model, the system may curate a paired dataset of protein structures and captions from both the PDB and UniProt databases. Caption information is collected for the structures used for the backbone diffusion model training, as well as the individual chains within these structures. For each structure, the system uses the PDB descriptive text as an overall caption. For each chain in a structure, the system obtains a caption by concatenating all available functional comments from UniProt. Structures containing more than 1000 residues are not used, corresponding to a minority (10%) of all structures. The final set used to train and validate the caption model contains approximately 45 thousand captions, including those from both PDB and UniProt. The splits used for training are completely random. The small size of the dataset constrained architecture choices to those with relatively few free parameters.

To build the caption model, the system may leverage a pretrained language model and a pretrained protein encoder. For example, the pretrained language model is the GPT-Neo 125 million parameter model. The system also leverages the pretrained graph neural network encoder, the protein structure classification model introduced above, to encode protein backbones. Analogously to the choice of the language model, the purpose of the structure encoder is to start The protein caption model with semantic knowledge of protein structure. To condition the autoregressive language model, GPT-Neo, pseudotokens are formed from structures using the ProClass encoder and prepended to the caption as context.

In one or more embodiments, the protein caption model connects a pretrained graph neural network encoder to an autoregressive language model trained on a large data corpus including scientific documents. Conditioning is achieved with pseudotokens generated from encodings of protein complex 3D backbone coordinates (batch size B, number of residues N, embedding dimension H) and a task token indicating whether a caption describes the whole complex or a single chain. The R relevant pseudotokens for each caption, consisting of the chain/structure residue tokens and the task token, are passed to the language model along with the caption. When used in the forward mode, the protein caption model can describe the protein backbone by outputting the probabilities of each word in the language model's vocabulary of size V for each of the L tokens of a caption. When used in conjunction with the prior model, it can be used for text to protein backbone synthesis. In training, the protein caption model uses a masked cross entropy loss applied only to the caption logits.

The system may perform embedding of the task, caption, and structure data into a shared tensor representation for input to the language model. Captions and task tokens are encoded using a modified version of the GPT-Neo tokenizer, whose vocabulary may be augmented with a special token to distinguish between prediction tasks involving single chains and those relating to entire structures. Structure inputs are converted into pseudotokens with the same shape as text embeddings through the graph neural network encoder of the pre-trained protein caption model. The task, structure, and caption embeddings are concatenated into a representation that is passed to the language model to obtain logits representing the probabilities of caption tokens. The model is trained on a standard masked cross entropy loss of the caption.

Structure encoding in the protein caption model relies on a pretrained classification model. This classifier model may be a GNN with multiple heads to extract different class information, as described previously. The GNN portion of the classifier network is used to obtain embeddings of each residue in the latent space of the classifier, with the intent that the pre-trained classifier weights should help the protein caption model learn the relationship between structures and captions. Besides the 3D information of the atoms in each structure, the diffusion timestep (noise level) is input to the GNN via a Fourier featurization layer which converts the diffusion time to a vector with the same dimension as the GNN node embedding space using randomly chosen frequencies between 0 and 16. To allow for the protein caption model to learn the optional use of sequence information, in 25% of the training data sequences are randomly passed along with structures. In these cases, the amino acid information for each residue is converted through a single embedding layer with output size equal to that of the GNN node embedding space dimension, then added to the time step vector.

Task tokens are added to the model to allow for captions of both single chain and full complex captions. For the prediction of UniProt captions describing single chains within structures, only the embeddings of the residues in the relevant chain are passed to the language model. For the prediction of the PDB captions related to entire structures, all residue embeddings are passed. In addition, a linear layer is added after the classification model embeddings to go between the classification model latent space and the embedding space of the language model, which are of different dimensionality.

Finally, in order to help the model distinguish between PDB and UniProt prediction tasks, the encodings of the entire structures are each prepended with an embedding vector of a newly defined PDB marker token. The system normalizes the components of all structure vectors such that each one has zero mean and unit variance.

In summary, the protein caption model architecture consists of a pre-trained GNN model for structure embedding and a pre-trained language model for caption embedding, with a learnable linear layer to interface between the two and a learnable language model head to convert the raw language model outputs to token probabilities.

The system trains the protein caption model to be compatible with conditional generation using the structural diffusion prior model. Like the other conditional models in this paper, each structure is noised according to the schedule of the structural diffusion model. During the protein caption model training, the graph neural network encoder weights from the pre-trained classification model are frozen. As the system adds a <|PDB|>task token to the GPT-Neo vocabulary to cue the model to predict whole complex captions from the PDB, the system allows the language model to learn in order to optimize the encoding of this new token and refine the embeddings of existing ones.

### Low-Temperature Sampling

In one or more implementations, low-temperature sampling implements a hybrid SDE combining temperature-dependent reverse-time SDE and modified Langevin dynamics with an equilibration rate.

Maximum likelihood training of generative models enforces a tolerable probability of all datapoints and, as a result, misspecified or low-capacity models fit by maximum likelihood will typically be overdispersed. This can be understood through the perspective that maximizing likelihood is equivalent to minimizing the KL divergence from the model to the data distribution, which is the mean-seeking and mode-covering direction of KL divergence.

To mitigate overdispersion in generative models, it is common practice to introduce modified sampling procedures that increase sampling of high-likelihood states (mode emphasis, precision) at the expense of reduced sample diversity (mode coverage, recall).

Here a novel algorithm for low-temperature sampling from diffusion models is disclosed. The novel algorithm leverages two concepts, explained in the next two sections. 1. Upscaling the score function of the reverse SDE is insufficient to properly re-weight populations in a temperature perturbed distribution. 2. Annealed Langevin dynamics can sample from low temperature distributions if given sufficient equilibration time.

### Reverse-Time Sde

In the isotropic Gaussian case, to determine how the Reverse-Time SDE can be modified to enable (approximate) low temperature sampling, it is helpful to first consider a case that can be treated exactly: transforming a Gaussian data distribution [symbol] (x₀; μ_{data}, σ_{data}²) to a Gaussian prior [symbol] (x₁; 0, σₚᵣᵢₒᵣ²).

Under the Variance-Preserving diffusion, the time-dependent marginal density will be given by:

$${p}_{t} ( x ) = 𝒩 ( x ; {α}_{t} {μ}_{data} , {{α}_{t}}^{2} {{σ}_{data}}^{2} + ( 1 - {{α}_{t}}^{2} ) {{σ}_{prior}}^{2} ) ,$$

which means that the score function sₜ will be:

$${s}_{t} {=}^{Δ} {∇}_{x} log {p}_{t} ( x ) = \frac{{α}_{t} {μ}_{data} - x}{{{α}_{t}}^{2} {{σ}_{data}}^{2} + ( 1 - {{α}_{t}}^{2} ) {{σ}_{prior}}^{2}} .$$

Now, suppose the system wish to modify the definition of the time-dependent score function so that, instead of transitioning to the original data distribution, it transforms to the perturbed data distribution, i.e., so that it transitions to

$$\frac{1}{Z} {{p}_{0} ( x )}^{{λ}_{0}} .$$

For a Gaussian, unis operation will simply multiply the precision (or equivalently, divide the covariance) by the factor λ₀. The perturbed score function will therefore be:

$${s}_{t}^{perturb} = \frac{{α}_{t} {μ}_{data} - x}{{{α}_{t}}^{2} {{σ}_{data}}^{2} / {λ}_{0} + ( 1 - {{α}_{t}}^{2} ) {{σ}_{prior}}^{2}} .$$

Based on this, the score function can be expressed as a time-dependent rescaling of the original score function with scaling based on the ratios of the time-dependent inverse variances as:

$${s}_{t}^{perturb} = {s}_{t} \frac{( 1 - {{α}_{t}}^{2} ) {{σ}_{prior}}^{2} + {{α}_{t}}^{2} {{σ}_{data}}^{2}}{( 1 - {{α}_{t}}^{2} ) {{σ}_{prior}}^{2} + {{α}_{t}}^{2} {{σ}_{data}}^{2} / {λ}_{0}} .$$

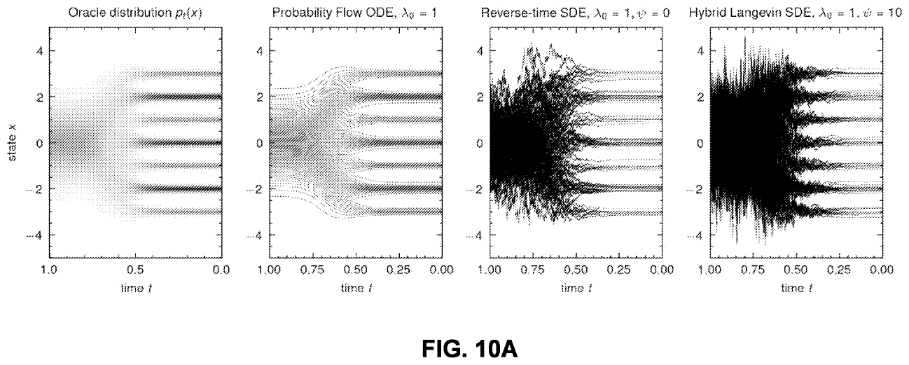

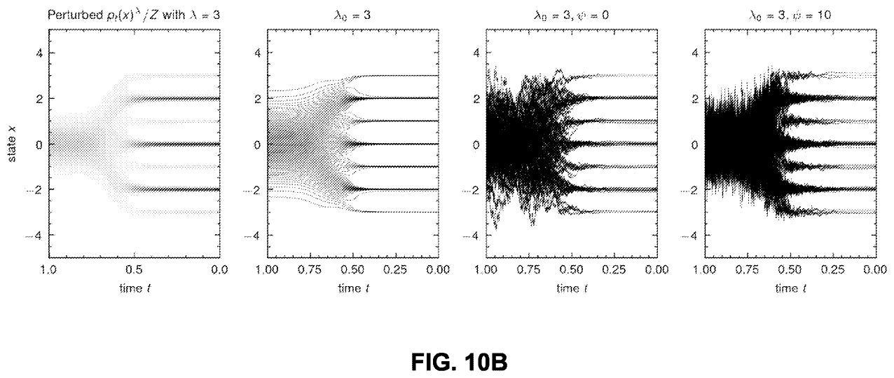

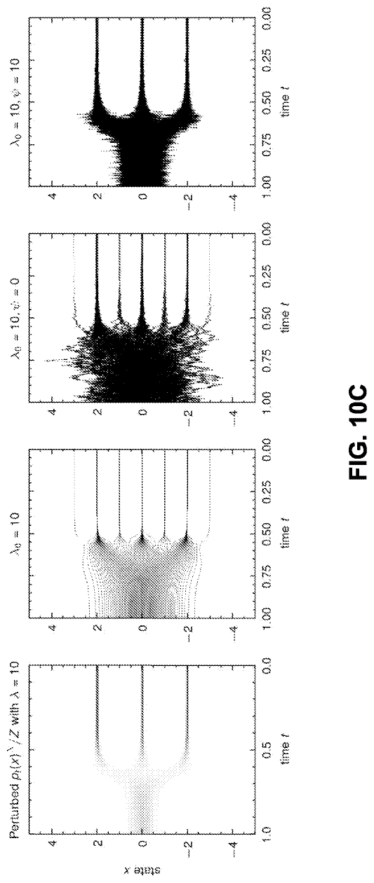

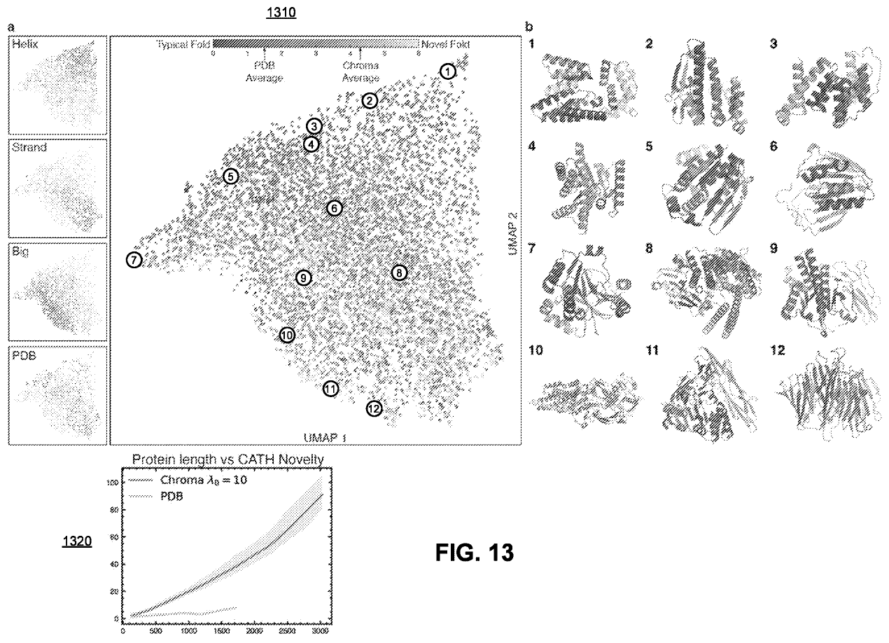

FIGS. 10A-10C illustrate Hybrid Langevin SDE to sample from temperature-perturbed distributions. The marginal densities of the diffusion process pₜ(x) (left) gradually transform between a toy 1D data distribution at time t=0 and a standard normal distribution at time t=T. Reweighting the distribution by inverse temperature (FIGS. 13B & 13C) will both concentrate and reweight the population distributions. The annealed versions of the reverse-time SDE and Probability Flow ODEs (middle columns) can concentrate towards local optima but do not correctly reweight the relative population occupancies. Adding in Langevin dynamics with the Hybrid Langevin SDE(right column) increases the rate of equilibration to the time-dependent marginals and, when combined with low temperature rescaling, successfully reweights the populations (right graph of FIG. 10C).

To achieve a particular inverse temperature Ao for the data distribution, the score function can be rescaled by the time-dependent factor:

$${λ}_{t} = \frac{( 1 - {{α}_{t}}^{2} ) {{σ}_{prior}}^{2} + {{α}_{t}}^{2} {{σ}_{data}}^{2}}{( 1 - {{α}_{t}}^{2} ) {{σ}_{prior}}^{2} + {{α}_{t}}^{2} {{σ}_{data}}^{2} / {λ}_{0}} \approx \frac{{λ}_{0}}{{{α}_{t}}^{2} + ( 1 - {{α}_{t}}^{2} ) {λ}_{0}} ,$$

where in the last step, σ_{data}²=σₚᵣᵢₒᵣ² is assumed. So one interpretation of the previously observed insufficiencies of low-temperature sampling based on score-rescaling is that they were hampered by uniform rescaling of the score function in time instead of in a way that accounts for the shift of influence between the prior and the data distribution.

To achieve temperature-adjusted reverse time SDE, the reverse-time SDE is modified by rescaling the score function with the above time-dependent temperature rescaling as:

$$dx = ( - \frac{1}{2} x - {λ}_{t} {RR}^{T} {∇}_{x} log {p}_{t} ( x ) ) {β}_{t} dt + \sqrt{{β}_{t}} R d \overline{w} = ( - \frac{1}{2} x - {λ}_{t} \frac{{α}_{t} {\hat{x}}_{θ} ( x , t ) - x}{1 - {{α}_{t}}^{2}} ) {β}_{t} dt + \sqrt{{β}_{t}} R d \overline{w} .$$

To achieve a temperature-adjusted probability flow ODE, the probability flow ODE can be rescaled as:

$$\frac{dx}{dt} = - \frac{{β}_{t}}{2} ( x + {λ}_{t} {RR}^{T} {∇}_{x} log {p}_{t} ( x ) ) = \frac{{β}_{t}}{2} ( \frac{{α}_{t} - {λ}_{t} - 1}{1 - {{α}_{t}}^{2}} - {\hat{x}}_{θ} ( x , t ) \frac{{λ}_{t} {α}_{t}}{1 - {{α}_{t}}^{2}} ) .$$

The rescaling rationale was derived by considering a unimodal Gaussian, which has the property that the score of the perturbed diffusion can be expressed as a rescaling of the learned diffusion. The above dynamics drive towards local maxima but do not reweight populations based on their relative probability. Accordingly, the low-temperature sampling algorithm incorporates an equilibration process that can be arbitrarily mixed in with the non-equilibrium reverse dynamics.

### Annealed Langevin Dynamics Sde

Instead of reversing the forwards time diffusion in a non-equilibrium manner, the low-temperature sampling algorithm can also leverage the learned time-dependent score function V̅ₓ log pₜ(x), as expressed in terms of the optimal denoiser \hat{x}_{θ}(x, t), to do slow, approximately equilibrated sampling with annealed Langevin dynamics.

The annealed Langevin dynamics is recast in continuous time with the SDE:

$$dx = - \frac{{β}_{t} Ψ}{2} {RR}^{T} {∇}_{x} log {{p}_{t} ( x )}^{{λ}_{0}} dt + \sqrt{{β}_{t} Ψ} R d \overline{w} = - \frac{{β}_{t} Ψ}{2} {λ}_{0} {RR}^{T} {∇}_{x} log {p}_{t} ( x ) dt + \sqrt{{β}_{t} Ψ} R d \overline{w}$$

where Ψ is an equilibration rate scaling the amount of Langevin dynamics per unit time. As Ψ→∞, the system will instantaneously equilibrate in time, constantly adjusting to the changing score function. These parameters can be set by considering a single Euler-Maruyama integration step in reverse time with step size

$$\frac{1}{T}$$

where T is the total number of steps:

$${x}_{t - \frac{1}{T}} ← {x}_{t} + \frac{{β}_{t} Ψ}{2 T} {λ}_{0} {RR}^{T} {∇}_{x} log {p}_{t} ( x ) + \sqrt{\frac{{β}_{t} Ψ}{T}} R ϵ , ϵ \sim 𝒩 ( 0 , I ) ,$$

which is precisely preconditioned Langevin dynamics with step size

$$\frac{{β}_{t} Ψ}{T} .$$

For a sufficiently small interval (t−dt, t), the system can keep the target density approximately fixed while increasing T to do an arbitrarily large number of Langevin dynamics steps, which will asymptotically equilibrate to the current density log pₜ(x).

### Hybrid Langevin-Reverse Time Sde

The low-temperature sampling algorithm may combine the annealed Reverse-Time SDE and the Langevin Dynamics SDE into a hybrid SDE that combines both dynamics. Denoting the inverse temperature as λ₀ and the ratio of the Langevin dynamics to convention dynamics as Ψ, the hybrid SDE can be expressed as:

$$dx = ( - \frac{1}{2} x - ( {λ}_{t} + \frac{{λ}_{0} Ψ}{2} ) {RR}^{T} {∇}_{x} log {p}_{t} ( x ) ) {β}_{t} dt + \sqrt{{β}_{t} ( 1 + Ψ )} R d \overline{w} = ( - \frac{1}{2} x - ( {λ}_{t} + \frac{{λ}_{0} Ψ}{2} ) {RR}^{T} \frac{{( {RR}^{T} )}^{- 1}}{1 - {{α}_{t}}^{2}} ( {α}_{t} {\hat{x}}_{θ} ( x , t ) - x ) ) {β}_{t} dt + \sqrt{{β}_{t} ( 1 + Ψ )} R d \overline{w} = ( - \frac{1}{2} x - ( {λ}_{t} + \frac{{λ}_{0} Ψ}{2} ) \frac{{α}_{t} {\hat{x}}_{θ} ( x , t ) - x}{1 - {{α}_{t}}^{2}} ) {β}_{t} dt + \sqrt{{β}_{t} ( 1 + Ψ )} R d \overline{w} .$$

where, when the scaling terms are set to unity, the standard reverse-time SDE is recovered.

In one or more generalized embodiments, the low-temperature sampling algorithm differentially scales the reverse-time SDE and/or the annealed Langevin Dynamics SDE. In such embodiments, the reverse-time SDE may be scaled by a first time-dependent factor with the annealed Langevin Dynamics SDE scaled by a second time-dependent factor. One or both of the time-dependent factors may be based on the inverse temperature, the equilibration rate, or some combination thereof. The inverse temperature and/or the equilibration rate may themselves be dependent on a state of the protein backbone on the time continuum.

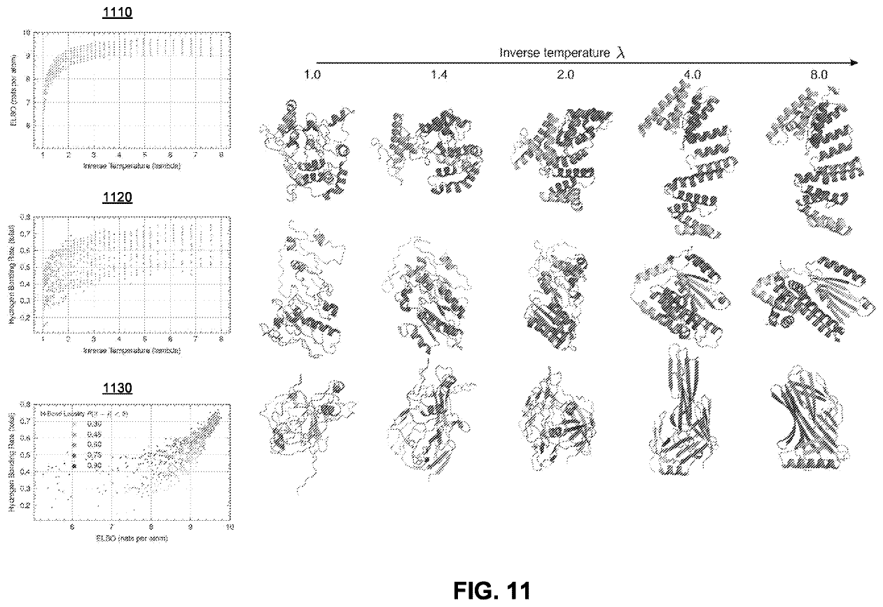

FIG. 11 illustrates representative samples identified using this modified SDE for low-temperature sampling. Generally, low-temperature sampling drives towards high-likelihood states with increased secondary structure content. Increasing the inverse temperature increases the likelihood (ELBO) for unconditional samples from the backbone diffusion model (Graph 1110). These high-likelihood states exhibit increased rates of backbone hydrogen bonding that underlie secondary structure (Graph 1120). Likewise, the ELBO is strongly associated with the hydrogen bonding rates (Graph 1130). These relationships can be seen within the evolution of single samples under fixed random seeds (each row of sampled backbones), where structures sampled at higher inverse temperature have increased secondary structure content and tighter packing as compact, globular folds.

In additional embodiments, while the Hybrid Langevin-Reverse Time SDE can do an arbitrarily large amount of Langevin dynamics per time interval which would equilibrate asymptotically in principle, these dynamics will still inefficiently mix between basins of attraction in the energy landscape when 0<t<<1. The system can further implement simulated tempering or parallel tempering, which would aid in deriving an augmented SDE system with auxiliary variables for the temperature and/or copies of the system at different time points in the diffusion.

### Example Results

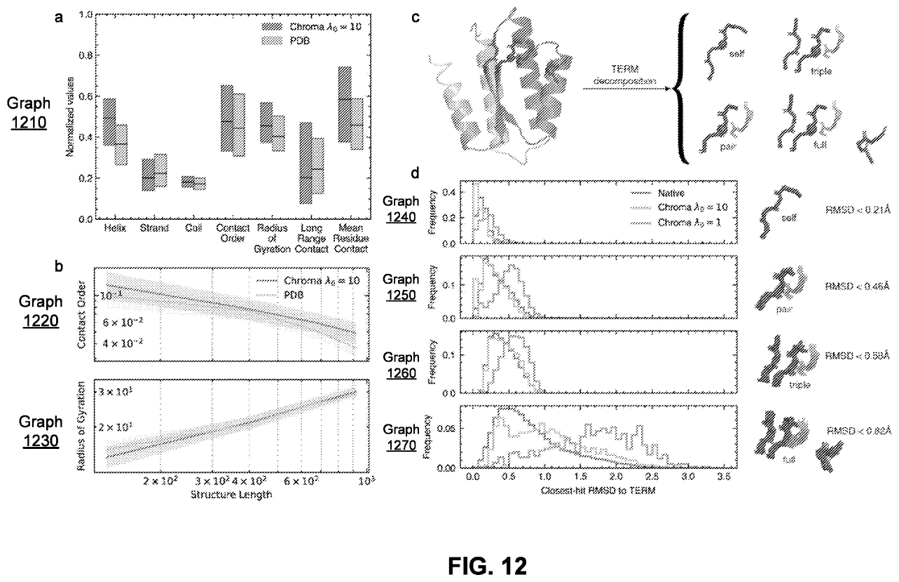

FIG. 12 illustrates various structural characteristics of synthetic protein designs generated with the diffusion model, according to one or more example implementations. As shown in Graph 1210, across a set a set of 10,000 single chains, samples from the diffusion model have structural properties that are similar to natural protein structures from the Protein Data Bank (PDB), including secondary structure utilization and length-normalized contact order, radius of gyration, and contact density statistics. Low-temperature samples from the diffusion model tend to favor helices over strands and are more compact than those found in the PDB. As shown in Graphs 1220 and 1230, the synthetic protein designs reproduce length-dependent scaling of contact order and radius of gyration, similar to proteins found in the PDB.

On the right side of FIG. 12, illustrated is a visual depiction of a tertiary motifs (referred to as “TERMs”) decomposition. The distribution of closest-match RMSD for TERMs of increasing order originating from native or Chroma-generated backbones (with inverse temperature λ0 being 1 or 10). Diffusion-generated protein backbones are designable by a variety of computational metrics.

FIG. 13 illustrates synthetic protein designs generated with the diffusion model, according to one or more example implementations. The synthetic protein designs span natural protein space while also frequently demonstrating high novelty. In Graph 1310, proteins from the PDB and the synthetic proteins generated by the diffusion model are featurized with 31 global-fold descriptors derived from knot theory and are embedded into two dimensions using Uniform Manifold Approximation and Projection (UMAP). The large figure is colored by the CATH coverage novelty measure normalized by protein length. Structural novelty was assessed by counting the number of CATH domains needed to achieve a greedy cover at least 80% of residues with TM>0.5. On average the diffusion model (referred to as “Chroma”) needs 4.3 CATH domains per 200 amino acids to cover 80% of its residues while structures from the PDB need only 1.6.

Of note, the synthetic proteins designed by the diffusion model are structurally more diverse and novel compared to structures from the PDB (regardless of protein length). The line represents the median value and is bounded by first (25%) and third (75%) quartile bands. The 4 smaller UMAP plots demonstrate the structure of the embedding by highlighting populations of structures that are mainly helices, strands, large (more than 500 residues), or natural proteins. The panel labeled PDB shows the distribution of natural proteins used to train the model.

On the right-side of the figure, twelve synthetic proteins are shown that were generated by the diffusion model, as a representative set across the embedding space. The twelve synthetic proteins all demonstrate a high novelty score (numbered in the embedding plot). The highlighted structures all have a novelty score of at least one standard deviation greater than the PDB.

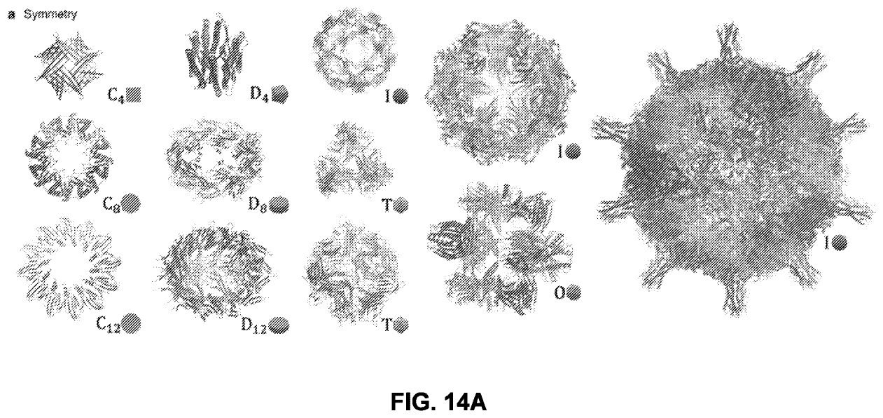

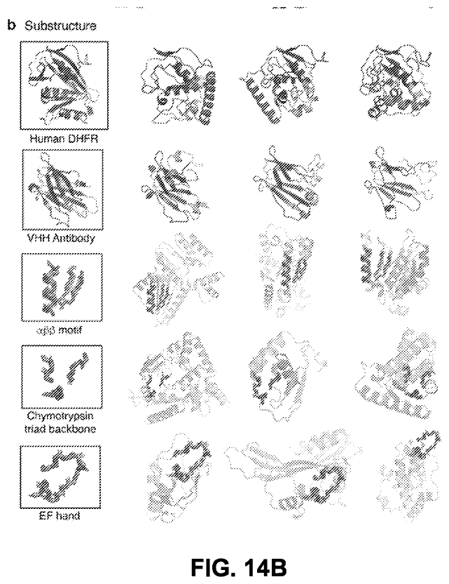

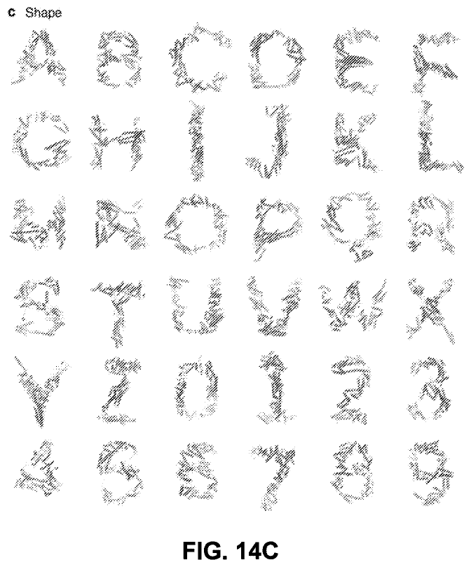

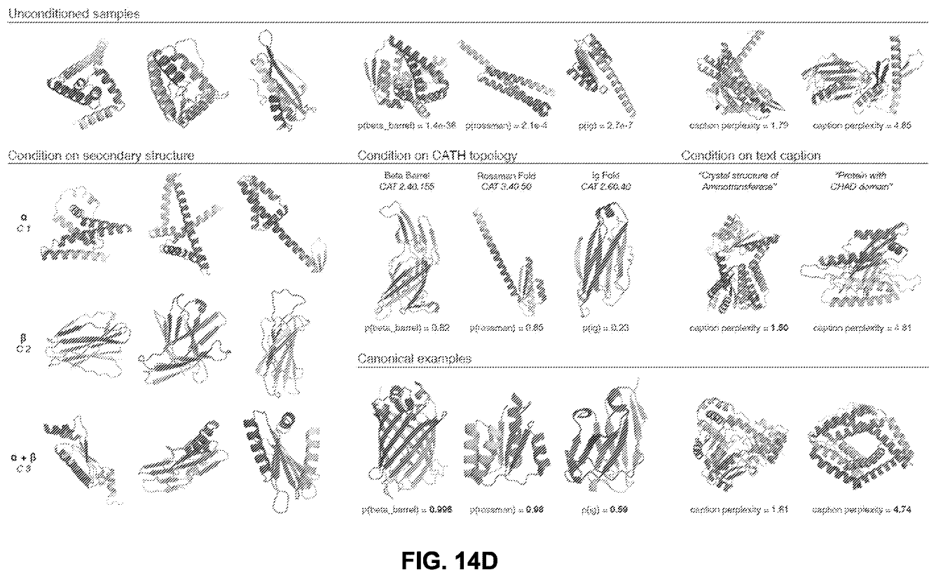

FIGS. 14A-D illustrate example synthetic protein designs satisfying varying design conditions, according to one or more example implementations. Symmetry, substructure, and shape conditioning enable geometric molecular programming.

FIG. 14A illustrates conditioning on arbitrary symmetry groups is possible by symmetrizing gradient, noise, and initialization through the sampling process. Cyclic Cn, dihedral Dn, tetrahedral T, octahedral O, and icosahedral I symmetries can produce a wide variety of possible homomeric complexes. The rightmost protein complex contains 60 subunits and 96,000 total residues.

FIG. 14B illustrates conditioning on partial substructure (monochrome) enables protein “infilling” or “outfilling.” The top two rows illustrate regeneration (color) of half of a protein (enzyme DHFR, first row) or CDR loops of an antibody (second row). The bottom three rows show conditioning on a pre-defined motif; order and matching location of motif segments is not pre-specified here.

FIG. 14C illustrates conditioning on arbitrary volumetric shapes by using gradients derived from Optimal Transport. Here, synthetic protein designs were conditioned to have backbone configurations subject to the complex geometries of the Latin alphabet and numerals.

FIG. 14D illustrates further conditioning based on other various design conditions. Protein structure classifiers and caption models can bias the sampling process towards user-specified properties. The top row shows example structures drawn unconditionally from the diffusion model. Below, models trained to predict protein semantics are used to conditionally sample structures with desired secondary structures, belonging to particular topologies, or corresponding to natural language captions. In each column, all conditional samples are drawn starting from the same random seed as the unconditional sample shown at the top of the column. The samples based on secondary structure conditioning show the impact of classifiers trained to predict mainly alpha, mainly beta, and mixed alpha-beta structures. In the columns with topology-conditioned samples, the classifier's predicted probabilities for the intended topology are indicated. Similarly, in the columns with samples based on text conditioning, the caption model's average perplexities are shown. For the topology and text caption columns, PDB structures are shown (“Canonical examples”) that exemplify the target condition.

### ADDITIONAL CONSIDERATIONS

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more subprocessing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated for the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A computer-implemented method comprising:
receiving a set of one or more design conditions that specify target characteristics of a synthetic protein;
defining a modular energy function as a composition of a diffusion energy component and one or more conditioner energy components, wherein the diffusion energy component determines an energy value based on a sampled state of the synthetic protein and a time step of the sampled state and each conditioner energy component determines an energy value based on the sample state of the synthetic protein and the target characteristic of each design condition; and
applying a diffusion model to determine a denoised protein backbone, wherein applying the diffusion model comprises, in each sampling step of a plurality of sampling steps:
transforming one prior sampled state of the synthetic protein from unconstrained space into constrained space based on the one or more design conditions,
denoising the prior sampled state in the constrained space, and
sampling a subsequent sampled state in the unconstrained space by applying a gradient of the modular energy function to the denoised prior sampled state in the constrained space;
wherein the final sampled state is a denoised protein backbone for the synthetic protein that satisfies the set of one or more design conditions.

2. The computer-implemented method of claim 1, wherein each design condition is either:
a restraint that reweights the modular energy function to bias for a target characteristic of the synthetic protein; or
a constraint that limits multidimensional protein space that defines possible states of the synthetic protein.

3. The computer-implemented method of any of claims 1-2, wherein the set of one or more design conditions one or more of:
a symmetry constraint that requires symmetry in the denoised protein backbone;
a substructure infilling restraint that biases towards particular substructures;
a shape constraint that requires a particular shape of the denoised protein backbone;
a distance constraint that requires a particular distance between at least two residues;
a substructure root mean squared deviation (RMSD) constraint that requires a structural motif to have a low RMSD;
a text caption restraint derived from a text input including one or more design conditions;
a sequence constraint that requires the denoised protein backbone to include a particular amino acid sequence;
a domain classifier constraint that inputs a target structure and outputs a functional characteristic required of the denoised protein backbone; and
a secondary structure constraint that requires a particular secondary structure to be present in the denoised protein backbone.

4. The computer-implemented method of any of claims 1-3, further comprising:
applying a sequence generation model to the denoised protein backbone to determine an amino acid sequence that folds into the denoised protein backbone.

5. The computer-implemented method of any of claims 1-4, wherein the diffusion model is further configured to output an amino acid sequence that is configured to structurally create the denoised protein backbone.

6. The computer-implemented method of any of claims 1-5, wherein an initial state is a base protein backbone to be modified by the diffusion model and is input with the set of one or more design conditions.

7. The computer-implemented method of any of claims 1-5, wherein an initial state is randomly sampled in multidimensional protein space.

8. The computer-implemented method of any of claims 1-7, wherein the plurality of sampling steps are discretized timesteps.

9. The computer-implemented method of claim 8, wherein the plurality of sampling steps includes 100 or more sampling steps.

10. The computer-implemented method of any of claims 1-9, wherein the set of one or more design conditions are derived from applying a natural language processing model to an input text query.

11. The computer-implemented method of any of claims 1-10, wherein, in each sampling step, sampling another sampled state comprises rescaling the modular energy function based on a time-dependent temperature.

12. The computer-implemented method of claim 11, wherein, in each sampling step, sampling another sampled state comprises applying a time-dependent Langevin dynamics equilibration rate.

13. The computer-implemented method of any of claims 1-12, further comprising:
initializing a first seed state and a second seed state that is different than the first seed state;
wherein applying the diffusion model comprises applying the diffusion model to the first seed state to determine a first denoised protein backbone and applying the diffusion model to the second seed state to determine a second denoised protein backbone.

14. The computer-implemented method of any of claims 1-13, further comprising:
receiving a second set of one or more design conditions that specify one or more modifications to the denoised protein backbone of the synthetic protein;
modifying the modular energy function to further comprise one or more conditioner energy components based on the second set of one or more design conditions; and
applying the diffusion model to modify the denoised protein backbone to satisfy the one or more modifications to the denoised protein backbone.

15. A non-transitory computer-readable storage medium storing instructions that, when executed by a computer processor, cause the computer processor to perform the computer-implemented method of any of claims 1-14.

16. A system comprising:
a computer processor; and
the non-transitory computer-readable storage medium of claim 15.

17. A non-transitory computer-readable storage medium storing a synthetic protein design that is generated by the computer-implemented method of any of claims 1-14.

18. A synthetic protein that is generated by a process comprising steps of:
determining a synthetic protein design that is generated by the computer-implemented method of any of claims 1-14; and
manufacturing the synthetic protein via cell expression.

19. A computer-implemented method for training a diffusion model, comprising:
accessing from a protein database a set of protein backbones;
generating a noised state for each protein backbone by transforming an initial state of the protein backbone with noise;
applying the diffusion model to the noised state for each protein backbone to predict a denoised state of the protein backbone;
determining a loss for each protein backbone as a difference between the denoised state and the initial state of the protein backbone; and
training the diffusion model as a neural network by adjusting one or more parameters of the diffusion model based on the losses.

20. The computer-implemented method of claim 19, wherein a protein backbone comprises three-dimensional coordinates for each heavy atom of amino acid residues in the protein backbone.

21. The computer-implemented method of any of claims 19-20, wherein generating the noised state for each protein backbone comprises, for each protein backbone:
selecting a random time step on a time continuum, wherein the initial state is at time step zero; and
adding an amount of Gaussian noise based on the random time step to the initial state of the protein backbone to generate the noised state.

22. The computer-implemented method of claim 21, wherein applying the diffusion model to the noised state for each protein backbone comprises:
predicting the amount of Gaussian noise added to generate the noised state based on the initial state and the random time step; and
removing the predicted amount of Gaussian noise from the noised state to generate the denoised state.

23. The computer-implemented method of any of claims 21-22, further comprising:
generating a second noised state for each protein backbone by:
selecting a second random time step on the time continuum, and
adding an amount of Gaussian noise based on the second random time step to the initial state of the protein backbone to generate the second noised state; and
applying the diffusion model to the second noised state for each protein backbone to predict a second denoised state of the protein backbone;
determining a second loss for each protein backbone as a difference between the second denoised state and the initial state of the protein backbone; and
wherein training the diffusion model is further based on the second losses.

24. The computer-implemented method of any of claims 19-23, wherein the loss for each protein backbone is based on a difference between coordinates of each heavy atom of amino acid residues in the denoised state and coordinates of each heavy atom of amino acid residues in the initial state.

25. The computer-implemented method of any of claims 19-24, further comprising:
filtering the protein database to deduplicate similar protein backbones.

26. The computer-implemented method of claim 25, wherein filtering the protein database to deduplicate similar protein backbones comprises:
determining a similarity score between a first protein backbone and a second protein backbone as a distance between coordinates of the first protein backbone and coordinates of the second protein backbone; and
removing the second protein backbone based on the similarity score being below a threshold.

27. The computer-implemented method of any of claims 19-26, further comprising:
filtering the protein database to obtain a high percentage of protein backbones of one type of protein.

28. A computer program product comprising:
a non-transitory computer-readable storage medium storing a diffusion model generated by the computer-implemented method of any of claims 19-27.

29. A non-transitory computer-readable storage medium storing a diffusion model generated by the computer-implemented method of any of claims 19-27.

30. A system comprising:
a computer processor; and
the non-transitory computer-readable storage medium of claim 29.

31. A computer-implemented method comprising:
receiving an input with an inverse temperature and an equilibration rate;
generating an energy function comprising a diffusion energy component which determines an energy value based on a state of a synthetic protein;
modifying a reverse-time dynamics function with a first scaling factor based on the inverse temperature, wherein the reverse-time dynamics function comprises a gradient of the energy function;
modifying a Langevin dynamics function with a second scaling factor based on the equilibration rate, wherein the Langevin dynamics function comprises the gradient of the energy function;
generating an aggregate dynamics function by combining the modified reverse-time dynamics function and the modified Langevin dynamics function;
initializing an initial state of a protein backbone comprising coordinates of heavy atoms of amino acids of a synthetic protein;
applying a diffusion model to the initial state to determine a denoised protein backbone, wherein applying the diffusion model comprises, in each sampling step of a plurality of sampling steps:
denoising one prior sampled state, and
sampling a subsequent sampled stated by applying the aggregate dynamics function to the denoised prior sampled state;
wherein the final sampled state is the denoised protein backbone for the synthetic protein.

32. The computer-implemented method of claim 31, wherein the inverse temperature is configured to drive the sampling towards high-likelihood regions of multidimensional protein space.

33. The computer-implemented method of any of claims 31-32, wherein the equilibration rate is a ratio of Langevin dynamics to conventional dynamics.

34. The computer-implemented method of any of claims 31-33, wherein the initial state is randomly sampled in multidimensional protein space.

35. The computer-implemented method of any of claims 31-34, wherein the plurality of sampling steps are discretized timesteps of a time continuum.

36. The computer-implemented method of claim 35, wherein the plurality of sampling steps includes 100 or more sampling steps.

37. The computer-implemented method of any of claims 31-36, further comprising:
applying a sequence generation model to the denoised protein backbone to determine an amino acid sequence that folds into the denoised protein backbone.

38. The computer-implemented method of any of claims 31-37, wherein the diffusion model is further configured to output an amino acid sequence that is configured to structurally create the denoised protein backbone.

39. The computer-implemented method of any of claims 31-38, wherein an initial sampled state is a base protein backbone to be modified by the diffusion model and is input with the inverse temperature and the equilibration rate.

40. The computer-implemented method of any of claims 31-38, wherein an initial sampled state is randomly sampled in multidimensional protein space.

41. A non-transitory computer-readable storage medium storing instructions that, when executed by a computer processor, cause the computer processor to perform the computer-implemented method of any of claims 31-40.

42. A system comprising:
a computer processor; and
the non-transitory computer-readable storage medium of claim 41.

43. A non-transitory computer-readable storage medium storing a synthetic protein design that is generated by the computer-implemented method of any of claims 31-40.

44. A synthetic protein that is generated by a process comprising steps of:
determining a synthetic protein design that is generated by the computer-implemented method of any of claims 31-40; and
manufacturing the synthetic protein via cell expression.
